(19) United States
(12) Patent Application Publication
(10) Pub. No.: US 2008/0039428 A1
(43) Pub. Date: Feb. 14, 2008

(54) Antiretroviral combination therapy

(75) Inventors: Graham Allaway, Darnestown, MD (US); Nicole Kilgore, Frederick, MD (US); Carl T. Wild, Gaithersburg, MD (US)

Correspondence Address: STERNE, KESSLER, GOLDSTEIN & FOX P.L.L.C., 1100 NEW YORK AVENUE, N.W., WASHINGTON, DC, 20005, US

(73) Assignee: Panacos Pharmaceuticals, Inc., Gaithersburg, MD (US)

(21) Appl. No.: 11/822,032

(22) Filed: Jun. 29, 2007

Related U.S. Application Data
(60) Provisional application No. 60/817,067, filed on Jun. 29, 2006.

(51) Int. Cl. A61K 31/661; A61K 31/145; A61K 31/34; A61K 31/44; A61K 31/47; A61P 31/12; A61K 31/505; A61K 31/519; A61K 31/52

(52) U.S. Cl. 514/99; 514/263.1; 514/274; 514/299; 514/307; 514/336; 514/357; 514/470; 514/557; 514/603; 514/789

(57) ABSTRACT

The present invention provides combinations comprising a viral maturation inhibitor and another therapeutically effective pharmaceutical agent. The invention is also directed to methods of treating a viral infection by administering such combinations.

### BACKGROUND OF THE INVENTION

### Field of the Invention

Human Immunodeficiency Virus (HIV) is a retrovirus that infects and invades cells of the immune system; it breaks down the body's immune system and renders the patient susceptible to opportunistic infections and neoplasms. The immune defect appears to be progressive and irreversible, with a high mortality rate that approaches 100% over several years.

The inherent tendency of HIV to mutate and become resistant to antiretroviral drugs remains a challenge to successful treatment. Patients with drug-resistant strains have an increasing risk of treatment failure with each subsequent treatment regimen. The concurrent use of multiple antiretroviral drugs has been reported to provide an improved virologic response and reduced probability for viral mutations.

Drug-resistant strains of HIV often appear over extended periods of time, even when the patient is on combination therapy. In some cases where an HIV strain is resistant to one drug in a therapeutic class, the strain will sometimes be cross-resistant to similar drugs in the same therapeutic class. Cross-resistance is a particular problem if a patient develops cross-resistance to a whole group of drugs. For example, if a patient has a cross-resistant strain exhibiting resistance to one non-nucleoside reverse transcriptase inhibitor (“NNRTI”), there is a risk that the patient's strain will be resistant to the entire NNRTI class.

Considerable progress has been made in the development of new antiretroviral drugs for treating HIV infection as well as in understanding how to use of existing drugs in combination therapies. Combinations of antiretroviral drugs are particularly effective in reducing viral load levels to undetectable levels while slowing the development of drug resistance.

Theoretically, each stage in the HIV life cycle presents opportunities to inhibit viral infection or replication. Phases of the HIV life cycle include:

1. Attachment,

2. Viral Fusion/Uncoating,

3. Viral Entry

4. Reverse Transcription,

5. RNaseH Degradation,

6. Second Strand Synthesis,

7. Migration to Nucleus,

8. Integration,

9. Latency,

10. Early Transcription,

11. Late Transcription,

12. RNA Processing,

13. Protein Synthesis,

14. Protein Glycosylation,

15. Assembly of Virion,

16. Viral Budding, and

17. Viral Maturation.

Until recently, all FDA drugs approved for HIV indications fell into two general classes: reverse transcriptase inhibitors and protease inhibitors. Generally, reverse transcriptase inhibitors block the conversion of HIV RNA to DNA. Generally, protease inhibitors prevent protease enzymes from cleaving viral subunits into various viral proteins. As more is learned about the HIV cellular life cycle, other promising targets have emerged. Certain stages of the HIV life cycle present preferred opportunities to inhibit viral infection or replication, including:

1. Virion-Cell Attachment,

2. Virion-Cell Fusion,

3. Entry,

4. Reverse Transcription,

5. Early Transcription,

6. Late Transcription,

7. Protein Synthesis,

8. Assembly,

9. Protein Glycosylation,

10. Viral Budding, and

11. Viral Maturation.

One new class of antiretrovirals is ‘fusion inhibitors.’ Fuzeon (enfuvirtide), which is designed to block HIV fusion to host cells, is the first FDA approved drug that acts at a target other than reverse transcriptase or protease.

Another new promising class of antiretroviral drugs is ‘maturation inhibitors’, which are believed to disrupt the formation of the HIV capsid protein and which lead to the creation of non-infectious virions. An example of a maturation inhibitor compound is 3-O-(3′,3′-dimethylsuccinyl)betulinic acid (“DSB”). As disclosed in U.S. Pat. No. 5,679,828, DSB is a potent inhibitor of HIV replication. As disclosed in US Patent Application 20050239748, one useful salt of DSB is the di-N-methyl-D-glucamine salt referred to herein as “DSBx2NmG.” In scientific literature, DSB and DSBx2NmG are occasionally referred to as “PA-457.” At other times in the scientific literature DSBx2NmG is referred to as “PA103001.”

Administration of a combination of drugs can reduce the risk of developing drug-resistant HIV. It is well known that combinations of drugs can potentially lead to unfavorable drug interactions including reduced efficacy of at least one of the components of the combination or deleterious effects on the patient's health.

There exists a therapeutic need for combinations comprising small molecule active pharmaceutical agents where the selection of the active pharmaceutical agents is not limited to reverse transcriptase inhibitors and protease inhibitors.

There exists a further therapeutic need for combinations comprising active pharmaceutical agents where the selection of the active pharmaceutical agents is not limited to reverse transcriptase inhibitors, protease inhibitors and the fusion inhibitors.

### BRIEF SUMMARY OF THE INVENTION

There is now provided a combination comprising a maturation inhibitor and an antiretroviral agent selected from the group consisting of entry inhibitors, reverse transcriptase inhibitors, integrase inhibitors, protease inhibitors, assembly inhibitors, budding inhibitors, and maturation inhibitors in amounts effective for treatment of HIV when used in a combination therapy.

There is now provided a combination comprising a maturation inhibitor and a secondary pharmaceutical agent selected from the group consisting of vaccines, gene therapy treatments, cytokines, TAT inhibitors, and immunomodulators in amounts effective for treatment of HIV when used in a combination therapy.

There is now provided a combination comprising a maturation inhibitor and a secondary pharmaceutical agent selected from the group consisting of antifungals, antibacterials, anti-neoplastics, anti-protozoals, DNA polymerase inhibitors, DNA synthesis inhibitors, anti-HIV antibodies, HIV antisense drugs, IL-2 agonists, α-glucosidase inhibitors, purine nucleoside phosphorylase inhibitors, apoptosis agonists, apoptosis inhibitors, and cholinesterase inhibitors in amounts effective for treatment of HIV when used in a combination therapy.

There is now further provided a combination comprising a maturation inhibitor and a secondary pharmaceutical agent selected from the group consisting of ribavirin, cellcept, amantadine, merimepodib, ANA975, CPG 10101, IDN-6556, BILN-2061, VRX-950, SCH6, SCH7, ACH-806, PSI-6130, Viramidine, Levovorin, valopicitabine, R803, HCV-086, HCV-796, JTK-003, JTK-109, NV-08, HCV-SM, R1626, ISIS014803, Hepex-C, HCV-AB68, Cicavir, HuMax-HepC, adefovir, adefovir dipivoxil, clevudine, telbivudine, Alamifovir, Remofovir, LB80380, NOV-205, EHT-899, HepeX-B, SpecifEx-HepB, LY-582563, EHT-899, and CellExSys in amounts effective for treatment of HIV when used in a combination therapy.

### BRIEF DESCRIPTION OF THE DRAWINGS/FIGURES

FIG. 1 depicts a median-effect plot of a combination of DSBx2NmG and AZT.

FIG. 2 depicts a median-effect plot of a combination of DSBx2NmG and Stavudine (D4T).

FIG. 3 depicts a median-effect plot of a combination of DSBx2NmG and lamivudine.

FIG. 4 depicts a median-effect plot of a combination of DSBx2NmG and nevirapine.

FIG. 5 depicts a median-effect plot of a combination of DSBx2NmG and indinavir.

FIG. 6 depicts a median-effect plot of a combination of DSBx2NmG and nelfinavir.

FIG. 7 depicts a median-effect plot of a combination of DSBx2NmG and saquinavir.

FIG. 8 depicts a median-effect plot of a combination of DSBx2NmG and ritonavir.

FIG. 9 depicts a median-effect plot of a combination of DSBx2NmG and emtricitabine.

FIG. 10 depicts a median-effect plot of a combination of DSBx2NmG and didanosine (DDI).

FIG. 11 depicts a median-effect plot of a combination of DSBx2NmG and abacavir.

FIG. 12 depicts a median-effect plot of a combination of DSBx2NmG and abacavir.

FIG. 13 depicts a median-effect plot of a combination of DSBx2NmG and efavirenz.

FIG. 14 depicts a median-effect plot of a combination of DSBx2NmG and tenofovir.

FIG. 15 depicts an isobologram of a combination of DSBx2NmG and AZT.

FIG. 16 depicts an isobologram of a combination of DSBx2NmG and stavudine (D4T).

FIG. 17 depicts an isobologram of a combination of DSBx2NmG and lamivudine.

FIG. 18 depicts an isobologram of a combination of DSBx2NmG and nevirapine.

FIG. 19 depicts an isobologram of a combination of DSBx2NmG and indinavir.

FIG. 20 depicts an isobologram of a combination of DSBx2NmG and nelfinavir.

FIG. 21 depicts an isobologram of a combination of DSBx2NmG and saquinavir.

FIG. 22 depicts an isobologram of a combination of DSBx2NmG and ritonavir.

FIG. 23 depicts an isobologram of a combination of DSBx2NmG and emtricitabine.

FIG. 24 depicts an isobologram of a combination of DSBx2NmG and atazanavir.

FIG. 25 depicts an isobologram of a combination of DSBx2NmG and emtricitabine.

FIG. 26 depicts an isobologram of a combination of DSBx2NmG and efavirenz.

FIG. 27 depicts an isobologram of a combination of DSBx2NmG and abacavir.

FIG. 28 depicts an isobologram of a combination of DSBx2NmG and

FIG. 29 depicts an isobologram of a combination of DSBx2NmG and didanosine (DDI).

### DETAILED DESCRIPTION OF THE INVENTION

The phrase “combination therapy” (or “co-therapy”) embraces the administration of a viral maturation inhibitor and a secondary pharmaceutical agent pharmaceutical agent as part of a specific treatment regimen intended to provide a beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected). “Combination therapy” generally is not intended to encompass the administration of two or more of these therapeutic agents as part of separate monotherapy regimens that incidentally and arbitrarily result in the combinations of the present invention. “Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. For example, one combination of the present invention comprises a reverse transcriptase inhibitor and a maturation inhibitor administered as separate agents at the same or different times or they can be formulated as a single, co-formulated pharmaceutical composition comprising the two compounds. As another example, a combination of the present invention comprises a reverse transcriptase inhibitor and a maturation inhibitor formulated as separate pharmaceutical compositions that can be administered at the same or different time. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, one component of a particular combination may be administered by intravenous injection while the other component(s) of the combination may be administered orally. The components may be administered in any therapeutically effective sequence.

“Combination therapy” also can embrace the administration of the therapeutic agents as described above in further combination with other biologically active ingredients (such as, but not limited to, a third and different therapeutic agent). “Combination therapy” also can embrace the administration of a therapeutic compound and a non-drug therapy, for example hyperthermia. Where the combination therapy comprises hyperthermia, the hyperthermic procedure can be conducted at any suitable time. In one embodiment, the administration of a combination comprising a maturation inhibitor and the hyperthermic procedure is designed to provide a beneficial effect from co-action of the combination.

The phrase “combination” embraces groups of at compounds or non-drug therapies useful as part of a combination therapy.

The term “viral entry” refers to several phases of the virus life cycle necessary for a virion to enter a target vesicle or cell, including attachment, fusion, penetration, and uncoating of the viral nucleocapsid.

The term “antiretroviral agent” refers to any process, action, application, therapy, or the like, wherein a human being is subject to medical aid with the object of improving the human's condition, directly or indirectly by inhibiting replication of a retrovirus.

The term “therapeutic effect” refers to some extent of relief of one or more of the symptoms of an HIV-related disorder. In reference to the treatment of HIV, a therapeutic effect refers to one or more of the following: 1) reduction in the number of infected cells; 2) reduction in the concentration of virions present in serum; 3) inhibition (i.e., slowing to some extent, preferably stopping) of rate of HIV replication; 4) increasing T-cell count; 5) relieving or reducing to some extent one or more of the symptoms associated with HIV; and 6) relieving or reducing the side effects associated with the administration of other antiretroviral agents.

“Therapeutically effective amount” is intended to qualify the amount required to achieve a therapeutic effect.

“EC50” refers to the drug concentration that results in a 50% reduction in virus replication.

Compounds useful in accordance with the present invention include pharmaceutically acceptable salt forms, prodrugs and stereoisomers thereof.

All references to dose of DSB herein are based on free acid equivalents.

With the intent of generating certain types of information relevant to the submission of an Investigational New Drug application (IND) or New Drug Application (NDA), certain combinations were tested to determine information such as co-action of certain components or synergistic effects of certain combinations.

Some embodiments of the present invention are focused on treating HIV infections. Other embodiments of the present invention are focused on treating disease states associated with HIV infection, for example, opportunistic infections. Other embodiments of the present invention are focused on treating HIV-Hepatitis co-infections, for example HIV-HCV co-infection or HIV-HBV co-infection.

There is now provided a combination comprising a maturation inhibitor and a secondary pharmaceutical agent selected from the group consisting of entry inhibitors, reverse transcriptase inhibitors, integrase inhibitors, protease inhibitors, assembly inhibitors, budding inhibitors, CCR5 antagonists, fusion inhibitors, and a second maturation inhibitor in amounts effective for treatment of HIV when used in a combination therapy. In one embodiment the maturation inhibitor is DSB.

There is now provided a combination comprising a maturation inhibitor and an antiretroviral agent selected from the group consisting of vaccines, gene therapy treatments, cytokines, TAT inhibitors, and immunomodulators in amounts effective for treatment of HIV when used in a combination therapy.

There is now provided a combination comprising a maturation inhibitor and an antiinfective agent selected from the group consisting of antifungals, antibacterials, anti-neoplastics, anti-protozoals, DNA polymerase inhibitors, DNA synthesis inhibitors, anti-HIV antibodies, HIV antisense drugs, IL-2 agonists, α-glucosidase inhibitors, purine nucleoside phosphorylase inhibitors, apoptosis agonists, apoptosis inhibitors, and cholinesterase inhibitors in amounts effective for treatment of HIV when used in a combination therapy.

There is now further provided a combination comprising a maturation inhibitor and an antiretroviral agent selected from the group consisting of ribavirin, cellcept, amantadine, merimepodib, ANA975, CPG 10101, IDN-6556, BILN-2061, VRX-950, SCH6, SCH7, ACH-806, PSI-6130, Viramidine, Levovorin, valopicitabine, R803, HCV-086, HCV-796, JTK-003, JTK-109, NV-08, HCV-SM, R1626, ISIS014803, Hepex-C, HCV-AB68, Cicavir, HuMax-HepC, adefovir, adefovir dipivoxil, clevudine, telbivudine, Alamifovir, Remofovir, LB80380, NOV-205, EHT-899, HepeX-B, SpecifEx-HepB, LY-582563, EHT-899, and CellExSys in amounts effective for treatment of HIV when used in a combination therapy.

One embodiment of the present invention provides a combination comprising an HIV maturation inhibitor and a CCR5 antagonist in amounts effective for treatment of HIV when used in a combination therapy. In some embodiments the CCR5 antagonist is maraviroc. In one embodiment the combination comprises 200-1000 mg DSBx2NmG and 150 mg maraviroc. Maraviroc is manufactured by Pfizer. In one embodiment the combination comprises 200-1000 mg DSBx2NmG and 300 mg maraviroc. Maraviroc is manufactured by Pfizer.

One embodiment of the present invention provides a combination comprising an HIV maturation inhibitor and a fusion inhibitor in amounts effective for treatment of HIV when used in a combination therapy. In some embodiments the fusion inhibitor is enfuvirtide. In one embodiment the combination comprises 200-1000 mg DSBx2NmG and 90 mg enfuvirtide. In one embodiment the combination comprises 200-1000 mg DSBx2NmG and 180 mg enfuvirtide.

One embodiment of the present invention provides a combination comprising an HIV maturation inhibitor and a protease inhibitor in amounts effective for treatment of HIV when used in a combination therapy. In some embodiments the maturation inhibitor is DSB. In some embodiments the protease inhibitor is selected from the group consisting of ritonavir, lopinavir, saquinavir, amprenavir (VX-478), fosamprenavir, nelfinavir (AG1343), tipranavir, indinavir, atazanavir (Nar-DG-35), TMC-126, darunavir (TMC-114), mozenavir (DMP-450), JE-2147 (AG1776), L-756423, KNI-272, DPC-681, DPC-684, telinavir (SC-52151), BMS 186318, droxinavir (SC-55389a), DMP-323, KNI-227, 1-[(2-hydroxyethoxy)methyl]-6-(phenylthio)-thymine, AG-1859, RO-033-4649, R-944, DMP-850, DMP-851, and brecanavir (GW640385). Preferred protease inhibitors for use in combination with DSBx2NmG include saquinavir, ritonavir, indinavir, nelfnavir, amprenavir, lopinavir, atazanavir, darunavir, brecanavir, fosamprenavir, and tipranavir.

In one embodiment the combination comprises 200-1000 mg DSBx2NmG and 100-1200 mg ritonavir. In another embodiment the combination comprises 200-1000 mg DSBx2NmG, 25-50 mg ritonavir and 100-250 mg lopinavir. In another embodiment the combination comprises 200-1000 mg DSBx2NmG, 25-50 mg ritonavir and 100-500 mg zidovudine. In another embodiment the combination comprises 200-1000 mg DSBx2NmG, 25-50 mg ritonavir and 100-500 mg didanosine. In another embodiment the combination comprises 200-1000 mg DSBx2NmG, 25-50 mg ritonavir and 25-150 mg stavudine. In another embodiment the combination comprises 200-1000 mg DSBx2NmG, 25-50 mg ritonavir and 100-500 mg lamivudine. In another embodiment the combination comprises 200-1000 mg DSBx2NmG, 25-50 mg ritonavir and 200-2500 mg indinavir. In another embodiment the combination comprises 200-1000 mg DSBx2NmG, 25-50 mg ritonavir and 150-2500 mg amprenavir. Ritonavir (Norvir or 10-Hydroxy-2-methyl-5-(1-methylethyl)-1-[2-(1-methylethyl)-4-thiazolyl]-3,6-dioxo-8,11-bis(phenylmethyl)-2,4,7, 12-tetraazatridecan-13-oic acid, 5-thiazolylmethyl ester, [5S-(5R*,8R*,10R*,11R*)]) is manufactured by Abbott Laboratories.

In one embodiment the combination comprises 200-1000 mg DSBx2NmG and lopinavir. Lopinavir ([1S-[1R*,(R*),3R*,4R*]]-N-[4[[(2,6-dimethylphenoxy)acetyl]amino]-3-hydroxy-5-phenyl-1-(phenylmethyl)pentyl]tetrahydro-alpha-(1-methylethyl)-2-oxo-1(2H)-pyrimidineacetamide) is manufactured by Abbott Laboratories.

In one embodiment the combination comprises 200-1000 mg DSBx2NmG and 1000-2400 mg saquinavir. In another embodiment the combination comprises 200-1000 mg DSBx2NmG, 1000-2400 mg saquinavir, and 50-200 mg ritonavir. In another embodiment the combination comprises 200-1000 mg DSBx2NmG and 750-2250 mg saquinavir mesylate. In another embodiment the combination comprises 200-1000 mg DSBx2NmG, 750-2250 mg saquinavir mesylate, and 50-200 mg ritonavir. Saquinavir mesylate (Invirase or N-tert-butyl-decahydro-2-[2(R)-hydroxy-4-phenyl-3(S)-[[N-(2-quinolylcarbonyl)-L-asparaginyl]amino]butyl]-(4aS,8aS)-isoquinoline-3(S)-carboxamide methanesulfonate) is manufactured by Roche Pharmaceuticals.

In one embodiment the combination comprises 200-1000 mg DSBx2NmG and 800-1200 mg amprenavir. Amprenavir (Agenerase or (3S)-tetrahydro-3-furyl N-[(1S,2R)-3-(4-amino-N-isobutylbenzenesulfonamido)-1-benzyl-2-hydroxypropyl]carbamate) is manufactured by GlaxoSmithKline Pharmaceuticals.

In one embodiment the combination comprises 200-1000 mg DSBx2NmG and 500-1500 mg fosamprenavir. In another embodiment the combination comprises 200-1000 mg DSBx2NmG and 500-1500 mg fosamprenavir sodium. In another embodiment the combination comprises 200-1000 mg DSBx2NmG and 500-1500 mg fosamprenavir calcium. In another embodiment the combination comprises 200-1000 mg DSBx2NmG, 500-1500 mg fosamprenavir sodium, and 50-250 mg ritonavir. In another embodiment the combination comprises 200-1000 mg DSBx2NmG, 500-1500 mg fosamprenavir calcium, and 50-250 mg ritonavir. Fosamprenavir calcium (Lexiva or (3S)-tetrahydrofuran-3-yl(1S,2R)-3-[[(4-aminophenyl)sulfonyl](isobutyl)amino]-1-benzyl-2-(phosphonooxy)propylcarbamate monocalcium) is manufactured by GlaxoSmithKline Pharmaceuticals.

In one embodiment the combination comprises 200-1000 mg DSBx2NmG and nelfinavir. In another embodiment the combination comprises 200-1000 mg DSBx2NmG and 500-2750 mg nelfinavir mesylate. Nelfinavir (Viracept or [3S-[2(2S*,3S*), 3a,4ab,8ab]]-N-(1,1-dimethylethyl)decahydro-2-[2-hydroxy-3-[(3-hydroxy-2-ethylbenzoyl)amino]-4-(phenylthio)butyl]-3-isoquinoline carboxamide mono-methanesulfonate) is manufactured by Pfizer.

In one embodiment the combination comprises 200-1000 mg DSBx2NmG and 500-2500 mg tipranavir. In another embodiment the combination comprises 200-1000 mg DSBx2NmG, 500-2500 mg tipranavir, and 100-500 mg ritonavir. Tipranavir (Aptivus) is manufactured by Boehringer-Ingelheim.

In one embodiment the combination comprises 200-1000 mg DSBx2NmG and 400-2400 mg indinavir. In another embodiment the combination comprises 200-1000 mg DSBx2NmG and 400-2400 mg indinavir sulfate ethanolate. In another embodiment the combination comprises 200-1000 mg DSBx2NmG, 400-2400 mg indinavir or a pharmaceutically acceptable salt thereof, and 100-500 mg ritonavir. Indinavir sulfate (Crixivan or [1(1S,2R),5(S)]-2,3,5-trideoxy-N-(2,3-dihydro-2-hydroxy-1H-inden-1-yl)-5-[2-[[(1,1-dimethylethyl)amino]carbonyl]-4-(3-pyridinylmethyl)-1-piperazinyl]-2-(phenylmethyl)-D-erythro-pentonamide sulfate) is manufactured by Merck & Co.

In one embodiment the combination comprises 200-1000 mg DSBx2NmG and 200-750 mg atazanavir. In another embodiment the combination comprises 200-1000 mg DSBx2NmG, 150-350 mg atazanavir, and 100-500 mg ritonavir. Atazanavir sulfate (Reyataz or (3S,8S,9S,12 S)-3,12-Bis(1,1-dimethylethyl)-8-hydroxy-4,11-dioxo-9-(phenylmethyl)-6-[[4-(2-pyridinyl)phenyl]methyl]-2,5,6,10,13-pentaazatetradecanedioic acid dimethyl ester, sulfate) is manufactured by Bristol-Myers Squibb Company.

In one embodiment the combination comprises 200-1000 mg DSBx2NmG and 500-1500 mg TMC-126. TMC-126 is manufactured by Tibotec Pharmaceuticals.

In one embodiment the combination comprises 200-1000 mg DSBx2NmG and 100-4000 mg darunavir. Darunavir (TMC-114) is manufactured by Tibotec Pharmaceuticals.

In one embodiment the combination comprises 200-1000 mg DSBx2NmG and 500-1500 mg mozenavir (DMP-450). In another embodiment the combination comprises 200-1000 mg DSBx2NmG and 500-1500 mg mozenavir dimesylate. Mozenavir (DMP-450 or (4R-(4α,5α,6β,7β)]hexahydro-5,6-dihydroxy-1,3-bis[((3-amino-phenyl)methyl)-4,7-bis(phenyl-methyl)-2H-1,3-diazepin-2-one) is manufactured by Triangle Pharmaceuticals, now owned by Gilead Sciences.

In one embodiment the combination comprises 200-1000 mg DSBx2NmG and JE-2147. JE-2147 (AG1776 or KNI-764) is manufactured by Agouron.

In one embodiment the combination comprises 200-1000 mg DSBx2NmG and L-756423. L-756423 (MK-944A) is manufactured by Merck & Co.

In one embodiment the combination comprises 200-1000 mg DSBx2NmG and DPC-681. DPC-681 is manufactured by Bristol-Myers Squibb Company.

In one embodiment the combination comprises 200-1000 mg DSBx2NmG and DPC-684. DPC-684 is manufactured by Bristol-Myers Squibb Company.

In one embodiment the combination comprises 200-1000 mg DSBx2NmG and 200-1000 mg brecanavir. In another embodiment the combination comprises 200-1000 mg DSBx2NmG, 300-750 mg brecanavir and 75-250 mg ritonavir. Brecanavir (GW640385) is manufactured by GlaxoSmithKline Pharmaceuticals.

In one embodiment the combination comprises 200-1000 mg DSBx2NmG and 500-1000 mg SC-52151. SC-52151 is manufactured by G.D. Searle, now owned by Pfizer.

In one embodiment the combination comprises 200-1000 mg DSBx2NmG and BMS-186318. BMS-186318 is manufactured by Bristol-Myers Squibb Company.

In one embodiment the combination comprises 200-1000 mg DSBx2NmG and SC-55389a. SC-55389a is manufactured by G.D. Searle, now owned by Pfizer.

In one embodiment the combination comprises 200-1000 mg DSBx2NmG and DMP-323. DMP-323 (XM-323 or [(4R-(4α,5α,6β,7β)]hexahydro-5,6-bis(hydroxy)-1,3-bis[(4-hydroxy-methyl)phenyl]methyl]-4,7-bis(phenylmethyl)-2H-1,3-diazepin-2-one) is manufactured by Avid Corporation.

In one embodiment the combination comprises 200-1000 mg DSBx2NmG and KNI-227. KNI-227 is manufactured by Pfizer.

In one embodiment the combination comprises 200-1000 mg DSBx2NmG and KNI-272. KNI-272 (kynostatin) is manufactured by Pfizer.

In one embodiment the combination comprises 200-1000 mg DSBx2NmG and 1-[(2-hydroxyethoxy)methyl]-6-(phenylthio)-thymine or an analog thereof.

In one embodiment the combination comprises 200-1000 mg DSBx2NmG and R82150. R82150 is being developed by Janssen Research Foundation.

In one embodiment the combination comprises 200-1000 mg DSBx2NmG and AG-1859. AG-1859 is manufactured by Pfizer.

In one embodiment the combination comprises 200-1000 mg DSBx2NmG and RO-033-4649. RO-033-4649 is manufactured by Hoffmann-La Roche Inc.

In one embodiment the combination comprises 200-1000 mg DSBx2NmG and Nar-DG-35. Nar-DG-35 is manufactured by Narhex.

In one embodiment the combination comprises 200-1000 mg DSBx2NmG and BMS-232632. BMS-232632 is manufactured by Bristol-Myers Squibb Company.

In one embodiment the combination comprises 200-1000 mg DSBx2NmG and GW-0385. GW-0385 is manufactured by GlaxoSmithKline Pharmaceuticals.

In one embodiment the combination comprises DSBx2NmG and a cyclic urea protease inhibitor. In some embodiments the cyclic urea protease inhibitor is DMP-850. In one embodiment the combination comprises 200-1000 mg DSBx2NmG and DMP-850. In some embodiments the cyclic urea protease inhibitor is DMP-851. In one embodiment the combination comprises 200-1000 mg DSBx2NmG and DMP-851. DMP-850 and DMP-851 are manufactured by DuPont Merck Pharmaceutical Company.

In one embodiment the combination comprises 200-1000 mg DSBx2NmG and PPL-100. PPL-100 is manufactured by Procyon Biopharma.

In one embodiment the combination comprises 200-1000 mg DSBx2NmG and PL-100. PL-100 is manufactured by Procyon Biopharma.

One embodiment of the present invention provides a combination comprising an HIV maturation inhibitor and a reverse transcriptase inhibitor in amounts effective for treatment of HIV when used in a combination therapy. In some embodiments the maturation inhibitor is DSB.

In some embodiments the reverse transcriptase inhibitor is selected from the group consisting of emtricitabine, capravirine, tenofovir, lamivudine, zalcitabine, delavirdine, nevirapine, didanosine, stavudine, abacavir, alovudine, zidovudine, racemic emtricitabine, emivirine, elvucitabine, brecanavir, DPC-083, amdoxovir, (−)-beta-D-2,6-diamino-purine dioxolane, MIV-210 (FLG), DFC (dexelvucitabine), dioxolane thymidine, Calanolide A, etravirine (TMC-125), L697639, atevirdine (U87201E), MIV-150, GSK-695634, GSK-678248, TMC-278, KP1461, KP-1212, lodenosine (FddA), 5-[(3,5-dichlorophenyl)thio]-4-isopropyl-1-(4-pyridylmethyl)imidazole-2-methanol carbamic acid, (−)-β-D-2,6-diaminopurine dioxolane, AVX-754, BCH-13520, BMS-56190 ((4S)-6-chloro-4-[(1E)-cyclopropylethenyl]-3,-4-dihydro-4-trifluoromethyl-2(1H)-quinazolinone), TMC-120, and L697639.

In some embodiments the reverse transcriptase inhibitor is a nucleoside reverse transcriptase inhibitor or nucleotide reverse transcriptase inhibitor selected from the group consisting of lamivudine, zidovudine, emtricitabine, abacavir, tenofovir, zalcitabine, didanosine, stavudine, atevirdine, alovudine, amdoxovir, brecanavir, racemic emtricitabine, dexelvucitabine, Dioxolane thymidine, elvucitabine, AVX-754, KP-1461, KP-1212, and MIV-210 (FLG). Preferred NRTIs for use in combination with DSBx2NmG include zidovudine (AZT), didanosine (ddI), zalcitabine (ddC), stavudine (d4T), tenofovir, lamivudine (3TC), abacavir, emitricitabine, and dexelvucitabine.

In some embodiments the reverse transcriptase inhibitor is a non-nucleoside analog reverse transcriptase inhibitor selected from the group consisting of delavirdine, nevirapine, capravirine, Calanolide A, emivirine, etravirine (TMC-125), L697639, atevirdine (U87201E) MIV-150, GSK-695634, GSK-678248, MV-057398, MV-057133, and TMC-278. Preferred NNRTIs for use in combination with DSBx2NmG include nevirapine, delaviradine, and etravirine.

In one embodiment the combination comprises 200-1000 mg DSBx2NmG and MV-057398. MV-057398 is manufactured by Medivir AB.

In one embodiment the combination comprises 200-1000 mg DSBx2NmG and MV-057133. MV-057133 is manufactured by Medivir AB.

In one embodiment the combination comprises 200-1000 mg DSBx2NmG and 20-550 mg L697639. L697639 is manufactured by Merck & Co.

In one embodiment the combination comprises 200-1000 mg DSBx2NmG and U87201E. U87201E (atevirdine) is manufactured by Upjohn, now owned by Pfizer.

In one embodiment the combination comprises 200-1000 mg DSBx2NmG and 500-1000 mg emivirine. Emivirine (MKC-442) is manufactured by Gilead Sciences.

In one embodiment the combination comprises 200-1000 mg DSBx2NmG and 5-30 mg alovudine. Alovudine is manufactured by Medivir.

In one embodiment the combination comprises 200-1000 mg DSBx2NmG and 200-500 mg zidovudine where the zidovudine may be delivered orally or intravenously. In another embodiment the combination comprises 200-1000 mg DSBx2NmG, 200-500 mg zidovudine, and 100-200 mg lamivudine where the zidovudine may be delivered orally or intravenously. In another embodiment the combination comprises 200-1000 mg DSBx2NmG, 200-500 mg zidovudine, 200-400 mg abacavir and 100-200 mg lamivudine where the zidovudine may be delivered orally or intravenously. Zidovudine (Retrovir or AZT or ZDV or 3′-azido-3′-deoxythymidine) is manufactured by GlaxoSmithKline Pharmaceuticals.

In one embodiment the combination comprises 200-1000 mg DSBx2NmG and (−)-β-D-2,6-diaminopurine dioxolane.

In one embodiment the combination comprises 200-1000 mg DSBx2NmG and 25-250 mg BMS-56190 (DPC-083 or (4S)-6-chloro-4-[(1E)-cyclopropylethenyl]-3,-4-dihydro-4-trifluoromethyl-2 (1H)-quinazolinone). BMS-56190 is manufactured by Bristol-Myers Squibb Company.

In one embodiment the combination comprises 200-1000 mg DSBx2NmG and DPC-961 ((4S)-6-chloro-4-(cyclopropylethynyl)-3,4-dihydro-4-(trifluoromethyl)-2 (1H)-quinazolinone). DPC-961 is manufactured by Bristol-Myers Squibb Company.

In one embodiment the combination comprises 200-1000 mg DSBx2NmG and DPC-961 ((S)-5,6-difluoro-4-cyclopropylethynyl-4-trifluoromethyl-3,4-dihydro-2(1H)-quinazolinone). DPC-963 is manufactured by Bristol-Myers Squibb Company.

In some embodiments, the reverse transcriptase inhibitor is a thiacytidine nucleoside analog, for example, emtricitabine. In one embodiment the combination comprises 200-1000 mg DSBx2NmG and 100-250 mg emtricitabine. Emtricitabine (Emtriva or 5-fluoro-1-(2R,5S)-[2-(hydroxymethyl)-1,3-oxathiolan-5-yl]cytosine) is manufactured by Gilead Sciences.

In some embodiments, the reverse transcriptase inhibitor is a dioxolane purine analog, for example, amdoxovir. In one embodiment the combination comprises 200-1000 mg DSBx2NmG and 200-600 mg amdoxovir. Amdoxovir is manufactured by RFS Pharma.

In one embodiment the combination comprises 200-1000 mg DSBx2NmG and 500-2500 mg capravirine. Capravirine (S-1153, AG-1549) is manufactured by Pfizer.

In one embodiment the combination comprises DSBx2NmG and tenofovir. In one embodiment the combination comprises 200-1000 mg DSBx2NmG and 150-400 mg tenofovir disoproxil fumarate. In another embodiment the combination comprises 200-1000 mg DSBx2NmG, 150-400 mg tenofovir disoproxil fumarate, and 160-300 mg emtricitabine. Tenofovir disoproxil fumarate (viread) is manufactured by Gilead Sciences. In some embodiments the combination comprises 200-1000 mg DSBx2NmG, 200-500 mg tenofovir, and 100-300 mg emtricitabine.

In one embodiment the combination comprises 200-1000 mg DSBx2NmG and 100-400 mg lamivudine. Lamivudine (Epivir or 3TC or (2R,cis)-4-amino-1-(2-hydroxymethyl-1,3-oxathiolan-5-yl)-(1H)-pyrimidin-2-one) is manufactured by GlaxoSmithKline Pharmaceuticals. In some embodiments the combination comprises 200-1000 mg DSBx2NmG, 75-250 mg zidovudine, and 100-400 mg lamivudine.

In one embodiment the combination comprises 200-1000 mg DSBx2NmG and 0.5-3.0 mg zalcitabine. zalcitabine (Hivid or 4-amino-1-beta-D-2′,3′-dideoxyribofuranosyl-2-(1H)-pyrimidone or 2′,3′-dideoxycytidine) is manufactured by Hoffmann-La Roche Pharmaceuticals.

In one embodiment the combination comprises 200-1000 mg DSBx2NmG and 100-400 mg nevirapine. Nevirapine (Viramune or 11-cyclopropyl-5,1,1-dihydro-4-methyl-6H-dipyrido[3,2-b:2′,3′-e][1,4]diazepin-6-one) is manufactured by Boehringer-Ingelheim.

In one embodiment the combination comprises 200-1000 mg DSBx2NmG and 100-500 mg didanosine. Didanosine (Videx or ddI or 2′,3′-dideoxyinosine) is manufactured by Bristol-Myers Squibb Company.

In one embodiment the combination comprises 200-1000 mg DSBx2NmG and 10-125 mg stavudine. Stavudine (Zerit or d4T or 2′,3′-didehydro-3′-deoxythymidine) is manufactured by Bristol-Myers Squibb Company.

In one embodiment the combination comprises 200-1000 mg DSBx2NmG and 25-250 mg dexelvucitabine. Dexelvucitabine is manufactured by Incyte.

In one embodiment the combination comprises 200-1000 mg DSBx2NmG and Elvucitabine. Elvucitabine (ACH-126443 or beta-L-Fd4C) is manufactured by Achillion Pharmaceuticals.

In one embodiment the combination comprises 200-1000 mg DSBx2NmG and 250-600 mg abacavir sulfate. In another embodiment the combination comprises 200-1000 mg DSBx2NmG, 400-800 mg abacavir, and 150-400 mg Epivir. Abacavir sulfate (Ziagen or (1S,cis)-4-[2-amino-6-(cyclopropylamino)-9H-purin-9-yl]-2-cyclopentene-1-methanol sulfate) is manufactured by GlaxoSmithKline Pharmaceuticals.

In another embodiment the combination comprises DSBx2NmG and a diarylpyrimidine reverse transcriptase inhibitor. In one embodiment the combination comprises 200-1000 mg DSBx2NmG and the diarylpyrimidine reverse transcriptase inhibitor etravirine. In one embodiment the combination comprises 200-1000 mg DSBx2NmG and the diarylpyrimidine reverse transcriptase inhibitor R278474. Etravirine (TMC-125 or R165335) is manufactured by Tibotec Pharmaceuticals.

In one embodiment the combination comprises 200-1000 mg DSBx2NmG and 50-900 mg TMC-120. TMC-120 (R147681)) is manufactured by Tibotec Pharmaceuticals.

In one embodiment the combination comprises 200-1000 mg DSBx2NmG and 200-500 mg delavirdine mesylate. In another embodiment the combination comprises 200-1000 mg DSBx2NmG, 200-500 mg delavirdine mesylate, and 100-300 mg zidovudine. Delavirdine (Rescriptor or piperazine, 1-[3-[(1-methyl-ethyl)amino]-2-pyridinyl]-4-[[5-[(methylsulfonyl)amino]-1H-indol-2-yl]carbonyl]-, monomethanesulfonate) is manufactured by Pfizer.

In one embodiment the combination comprises 200-1000 mg DSBx2NmG and DPC-817. DPC-817 (dexelvucitabine or D-D4FC) is manufactured by Pharmasset and Incyte.

In one embodiment the combination comprises 200-1000 mg DSBx2NmG and GS-7340. GS-7340 is manufactured by Gilead Sciences.

In one embodiment the combination comprises 200-1000 mg DSBx2NmG and GS-9005. GS-9005 is manufactured by Gilead Sciences.

In one embodiment the combination comprises 200-1000 mg DSBx2NmG and GW-5634. GW-5634 (GW-695634) is manufactured by GlaxoSmithKline Pharmaceuticals.

In one embodiment the combination comprises 200-1000 mg DSBx2NmG and SJ-3366. SJ-3366 is manufactured by Samjin Pharmaceutical Co.

In one embodiment the combination comprises 200-1000 mg DSBx2NmG and Phosphazid. Phosphazid is manufactured by Institute Biochemical Research.

In one embodiment the combination comprises 200-1000 mg DSBx2NmG and 200-1000 mg Phosphonovir (PZT or 3′-azido-2′, 3′dideoxythymine-5′-H-phosphonate). Phosphonovir is manufactured by Viscount Pharma.

In one embodiment the combination comprises 200-1000 mg DSBx2NmG and Thiovir. Thiovir (thiophosphonoformic acid) is manufactured by Adventrx Pharmaceuticals.

In one embodiment the combination comprises DSBx2NmG and a thiocarboxanilide nonnucleoside inhibitor. In some embodiments the thiocarboxanilide nonnucleoside inhibitor is UC-781. In one embodiment the combination comprises 200-1000 mg DSBx2NmG and UC-781. UC-781 is being developed by the University of Pittsburgh.

One embodiment of the present invention provides a combination comprising an HIV maturation inhibitor and a viral entry inhibitor in amounts effective for treatment of HIV when used in a combination therapy. In some embodiments the maturation inhibitor is DSB. In some embodiments, the viral entry inhibitor is an attachment inhibitor. In some embodiments, the viral entry inhibitor is a fusion inhibitor. In some embodiments, the viral entry inhibitor is a CD4 receptor binding inhibitor. In some embodiments, the viral entry inhibitor is a CD4 mimic. In some embodiments, the viral entry inhibitor is a gp120 mimic. In some embodiments, the viral entry inhibitor is a gp41 antagonist. In some embodiments, the viral entry inhibitor is a CD4 monoclonal antibody. In some embodiments, the viral entry inhibitor is a CCR5 antagonist. In some embodiments, the viral entry inhibitor comprises a sub-class of CCR5 antagonists, for example a zinc finger inhibitor. In some embodiments, the viral entry inhibitor is a CXCR4 coreceptor antagonist.

In some embodiments, the viral entry inhibitor is an attachment inhibitor selected from the group consisting of BMS-806, BMS-488043, BMS-378806, AK602, SP01A and Sifurvitide.

In one embodiment the combination comprises 200-1000 mg DSBx2NmG and BMS-043. BMS-043 is manufactured by Bristol-Myers Squibb Company.

In one embodiment the combination comprises 200-1000 mg DSBx2NmG and BMS-488043. BMS-488043 is manufactured by Bristol-Myers Squibb Company.

In one embodiment the combination comprises 200-1000 mg DSBx2NmG and BMS-378806. BMS-378806 is manufactured by Bristol-Myers Squibb Company.

In one embodiment the combination comprises 200-1000 mg DSBx2NmG and Sifurvitide. Sifurvitide is manufactured by FusoGen Pharmaceuticals.

In one embodiment the combination comprises 200-1000 mg DSBx2NmG and SP01A. SP01A is manufactured by Samaritan Pharmaceuticals.

In some embodiments, the viral entry inhibitor is a fusion inhibitor. In one embodiment, the fusion inhibitor is enfuvirtide. the In one embodiment the combination comprises 200-1000 mg DSBx2NmG and 50-150 mg enfuivirtide wherein the enfuivirtide is delivered subcutaneously. Enfuvirtide (Fuzeon or a peptide having the sequence CH3CO-Tyr-Thr-Ser-Leu-Ile-His-Ser-Leu-Ile-Glu-Glu-Ser-Gln-Asn-Gln-Gln-Glu-Lys-Asn-Glu-Gln-Glu-Leu-Leu-Glu-Leu-Asp-Lys-Trp-Ala-Ser-Leu-Trp-Asn-Trp-Phe-NH2) is manufactured by Roche Laboratories Inc. and Trimeris Inc.

In one embodiment the combination comprises 200-1000 mg DSBx2NmG and 5-200 mg T-1249 wherein the T-1249 is delivered subcutaneously.

One embodiment of the present invention provides a combination comprising an HIV maturation inhibitor and a gp41 antagonist in amounts effective for treatment of HIV when used in a combination therapy. In some embodiments the maturation inhibitor is DSB.

In one embodiment the combination comprises 200-1000 mg DSBx2NmG and NB-2. NB-2 (4-(2,5-dimethyl-1H-pyrrol-1-yl)-2-hydroxy-benzoic acid) is manufactured by Shibo-Kumar.

In one embodiment the combination comprises 200-1000 mg DSBx2NmG and NB-64. NB-64 (2-chloro-5-(1H-pyrrol-1-yl)-benzoic acid) is manufactured by Shibo-Kumar.

One embodiment of the present invention provides a combination comprising an HIV maturation inhibitor and a zinc finger inhibitor in amounts effective for treatment of HIV when used in a combination therapy. In some embodiments the maturation inhibitor is DSB. In some embodiments, the zinc finger inhibitor is a benzamidedisulfide, for example, ACH-0100703. In other embodiments, the zinc finger inhibitor is azodicarbonamide (ADA).

In one embodiment the combination comprises 200-1000 mg DSBx2NmG and ACH-0100703. ACH-0100703 is manufactured by Achillion Pharmaceuticals.

In some embodiments, the fusion inhibitor is a bicyclam.

In one embodiment the combination comprises 200-1000 mg DSBx2NmG and AMD-3100. AMD-3100 (JM-2987 or 1,1′-[1,4-Phenylenebis(methylene)]bis[1,4,8,11-tetraazacyclotetradecane]) is manufactured by AnorMED Inc.

In one embodiment the combination comprises 200-1000 mg DSBx2NmG and AMD-887. AMD-887 is manufactured by AnorMED Inc.

In one embodiment the combination comprises 200-1000 mg DSBx2NmG and AMD-070. AMD-070 is manufactured by AnorMED Inc.

In one embodiment the combination comprises 200-1000 mg DSBx2NmG and AMD-3451. AMD-3451 is manufactured by AnorMED Inc.

In some embodiments, the viral entry inhibitor is a CCR5 antagonist selected from the group consisting of monoclonal antibodies to CCR5, and a small molecule antagonists

In some embodiments, the CCR5 antagonist is a small molecule entry antagonist, for example, TAK-779, TAK-652, TAK-220, CCR5mAb004, SCH-351125, CMPD-167, and GW-873140.

In one embodiment the combination comprises 200-1000 mg DSBx2NmG and TAK-779. TAK-779 is manufactured by Takeda Pharmaceuticals.

In one embodiment the combination comprises 200-1000 mg DSBx2NmG and TAK-652. TAK-652 is manufactured by Takeda Pharmaceuticals.

In one embodiment the combination comprises 200-1000 mg DSBx2NmG and TAK-220. TAK-220 is manufactured by Takeda Pharmaceuticals.

In one embodiment the combination comprises 200-1000 mg DSBx2NmG and SCH-351125. SCH-351125 (SCH-C) is manufactured by Schering-Plough.

In one embodiment the combination comprises 200-1000 mg DSBx2NmG and 50-300 mg CMPD-167. CMPD-167 is manufactured by Merck & Co.

In one embodiment the combination comprises 200-1000 mg DSBx2NmG and 50-400 mg GW873140. GW873140 (AK602 or ONO4128) is being developed by GlaxoSmithKline.

In one embodiment the combination comprises 200-1000 mg DSBx2NmG and CCR5 mAb004. CCR5 mAb004 is manufactured by Human Genome Sciences.

In one embodiment the combination comprises 200-1000 mg DSBx2NmG and GBV-C. GBV-C is being developed by the University of Iowa.

In one embodiment the combination comprises 200-1000 mg DSBx2NmG and IC9564. IC9564 ((3R,4S)—N′-[N-3.beta.-Hydroxylup-20(29)-en-28-oyl]-8-aminooctanoyl]-4-amino-3-hydroxy-6-methylheptanoic acid) is being developed by Duke University.

In one embodiment the combination comprises 200-1000 mg DSBx2NmG and the CXCR4 antagonist is KRH-2731. KRH-2731 is manufactured by Kureha Corp.

In one embodiment the combination comprises 200-1000 mg DSBx2NmG and the CXCR4 antagonist is KRH-3955. KRH-3955 is manufactured by Kureha Corp.

In one embodiment the combination comprises 200-1000 mg DSBx2NmG and the CXCR4 antagonist is KRH-3140. KRH-3140 is manufactured by Kureha Corp.

In some embodiments, the entry inhibitor comprises a monoclonal antibody selected from the group consisting of 2D7mAb, PA8 mAb, PA9mAb, PA10mAb, PA11mAb, PA12mAb, and PA14mAb. TAK-779, TAK-652, TAK-220, SCH-351125, CMPD-167, UK427857 or GW-873140

In some embodiments, the entry inhibitor is a CCR5 inhibitor selected from the group consisting of maraviroc, aplaviroc, and vicriviroc.

In one embodiment the combination comprises 200-1000 mg DSBx2NmG and 150-500 mg maraviroc. Maraviroc (UK-427857 or 4,4-difluoro-N-[(1S)-3-{(1R,3s,5S)-3-[3-methyl-5-(propan-2-yl)-4H-1,2,4-triazol-4-yl]-8-azabicyclo[3.2.1]octan-8-yl}-1-phenylpropyl]cyclohexanecarboxamide) is manufactured by Pfizer.

In one embodiment the combination comprises 200-1000 mg DSBx2NmG and 200-800 mg aplaviroc. Aplaviroc (GSK-873140 or (4-(4-{[(3R)-1-butyl-3-[(R)-cyclohexylhydroxymethyl]-2,5-dioxo-1,4,9-triazaspiro[5.5]undecan-9-yl]methyl}phenoxy)benzoic acid)) is manufactured by GlaxoSmithKline.

In one embodiment the combination comprises 200-1000 mg DSBx2NmG and 5-50 mg vicriviroc. Vicriviroc (SCH-D or SCH-417690 or (4,6-dimethylpyrimidin-5-yl){4-[(3S)-4-{(1R)-2-methoxy-1-[4-(trifluoromethyl)phenyl]ethyl}-3-methylpiperazin-1-yl]-4-methylpiperidin-1-yl}methanone) is manufactured by Schering Plough.

In some embodiments, the viral entry inhibitor is a gp120 mimic. In one embodiment the combination comprises 200-1000 mg DSBx2NmG and SPC3.

In some embodiments, the viral entry inhibitor is a CD4 mimic. In one embodiment the combination comprises 200-1000 mg DSBx2NmG and PRO-542.

In one embodiment the combination comprises 200-1000 mg DSBx2NmG and 5-1250 mg PRO-542 where the PRO-542 may be delivered intravenously.

In some embodiments, the entry inhibitor is a CD4 binding inhibitor selected from the group consisting of BMS-488043, TNX-355, and Cosalane.

In one embodiment the combination comprises 200-1000 mg DSBx2NmG and Cosalane. Cosalane (NSC658586 or the ammonium salt of 3,3′-[4-(3β-5α)-cholestan-3-yl-1-butenylidene]bis[5-chloro-6-hydroxy-benzoic acid) is manufactured by Purdue Research Foundation.

In some embodiments, the entry inhibitor is a chemokine receptor inhibitor is selected from the group consisting of PRO 140, and Schering C (SCH-C).

In one embodiment the combination comprises 200-1000 mg DSBx2NmG and 50-800 mg SCH-C. SCH-C is manufactured by Schering-Plough.

In one embodiment the combination comprises 200-1000 mg DSBx2NmG and PRO 140. PRO 140 is manufactured by Progenics Pharmaceuticals.

In one embodiment the combination comprises 200-1000 mg DSBx2NmG and SCH-D. SCH-D is manufactured by Schering-Plough.

One embodiment of the present invention provides a combination comprising an HIV maturation inhibitor and an immunomodulator in amounts effective for treatment of HIV when used in a combination therapy. In some embodiments the maturation inhibitor is DSB. In some embodiments, the immunomodulator is selected from the group consisting of pentamidine isethionate, autologous CD8+ infusion, α-interferon immunoglobulins, thymic peptides, IGF-1, anti-Leu3A, autovaccination, biostimulation, extracorporeal photophoresis, cyclosporin, rapamycin, FK-565, FK-506, GCSF, GM-CSF, hyperthermia, isopinosine, IVIG, HIVIG, passive immunotherapy and polio vaccine hyperimmunization.

In one embodiment the combination comprises 200-1000 mg DSBx2NmG and bropirimine. Bropirimine is manufactured by Upjohn, now owned by Pfizer.

In one embodiment the combination comprises 200-1000 mg DSBx2NmG and Ampligen. Ampligen (poly I: poly C12U) is manufactured by Hemispherx Biopharma.

In one embodiment the combination comprises 200-1000 mg DSBx2NmG and anti-human alpha interferon antibody.

In one embodiment the combination comprises DSBx2NmG and a colony stimulating factor. In some embodiments the colony stimulating factor is a granulocyte colony stimulating factor, for example, Neupogen. In some embodiments the colony stimulating factor is a granulocyte macrophage colony stimulating factor, for example, Leucomax or sargramostim. In some embodiments the colony stimulating factor is a macrophage colony stimulating factor.

In one embodiment the combination comprises 200-1000 mg DSBx2NmG and 400-800 mg Neupogen wherein the Neupogen may be delivered subcutaneously or intravenously. Neupogen (Filgrastim or recombinant methionyl human granulocyte colony-stimulating factor or r-metHuG-CSF) is manufactured by Amgen.

In one embodiment the combination comprises 200-1000 mg DSBx2NmG and CL246738. CL246738 is also known as [3,6-bis(2-piperidinoethoxy)acridine trihydrochloride).

In one embodiment the combination comprises 200-1000 mg DSBx2NmG and Imreg-1. Imreg-1, a derivative of the immunostimulant Tyr1-Gly2-Gly3, is manufactured by Imreg Inc.

In one embodiment the combination comprises 200-1000 mg DSBx2NmG and Imreg-2. Imreg-2, a derivative of the immunostimulant Tyr1-Gly2-Gly3, is manufactured by Imreg Inc.

In one embodiment the combination comprises 200-1000 mg DSBx2NmG and diethydithiocarbamate.

In one embodiment the combination comprises 200-1000 mg DSBx2NmG and 6-10 MIU interleukin-2 wherein the interleukin-2 may be delivered intravenously or subcutaneously.

In one embodiment the combination comprises 200-1000 mg DSBx2NmG and an interferon.

In one embodiment the combination comprises 200-1000 mg DSBx2NmG and an α-interferon selected from the group consisting of peginterferon alfa 2a, peginterferon alfa 2b, and human leukocyte interferon-α.

In one embodiment the combination comprises 200-1000 mg DSBx2NmG and a peginterferon α-2a. Peginterferon α-2a (Pegasys) is manufactured by Hoffman-La Roche. In some embodiments the combination comprises 200-1000 mg DSBx2NmG, a peginterferon α-2a, and ribavirin.

In one embodiment the combination comprises 200-1000 mg DSBx2NmG and a peginterferon α-2b. Peginterferon α-2b (Viraferon Peg or Peg-Intron) is manufactured by Schering-Plough Corp.

In one embodiment the combination comprises 200-1000 mg DSBx2NmG and human leukocyte interferon-α.

In one embodiment the combination comprises 200-1000 mg DSBx2NmG and an interferon is selected from the group consisting of Sumiferon, MultiFeron, interferon-τ, and Reticulose.

In one embodiment the combination comprises 200-1000 mg DSBx2NmG and Sumiferon. Sumiferon is manufactured by Sumitomo Corp.

In one embodiment the combination comprises 200-1000 mg DSBx2NmG and MultiFeron. MultiFeron is manufactured by Viragen.

In one embodiment the combination comprises 200-1000 mg DSBx2NmG and interferon-τ.

In one embodiment the combination comprises 200-1000 mg DSBx2NmG and Reticulose. Reticulose is manufactured by Advanced Viral Research.

In one embodiment the combination comprises 200-1000 mg DSBx2NmG and Alferon LDO. Alferon LDO is manufactured by HemispheRx Biopharma.

In one embodiment the combination comprises DSB and a beta-interferon. In one embodiment the combination comprises 200-1000 mg DSBx2NmG and Interferon β-1b. Interferon β-1b (Betaseron) is manufactured by Berlex.

In one embodiment the combination comprises 200-1000 mg DSBx2NmG and inosine pranobex.

In one embodiment the combination comprises 200-1000 mg DSBx2NmG and methionine-enkephalin.

In one embodiment the combination comprises 200-1000 mg DSBx2NmG and muramyl-tripeptide.

In some embodiment the combination comprises DSB and a thymic peptide. In some embodiments the thymic peptide is selected from the group consisting of thymopentin, thymostimulin, thymic humoral factor γ-2, and thymosin α-1.

In one embodiment the combination comprises 200-1000 mg DSBx2NmG and Thymopentin. Thymopentin (TP-5 or Timunox) is manufactured by Janssen-Cilag.

In one embodiment the combination comprises 200-1000 mg DSBx2NmG and thymostimulin. Thymostimulin (TP-1) is manufactured by Ellen Pharmaceuticals

In one embodiment the combination comprises 200-1000 mg DSBx2NmG and thymic humoral factor γ-2. Thymic humoral factor γ-2 (THF-γ-2) is manufactured by Pharmacia & Upjohn, now owned by Pfizer.

In one embodiment the combination comprises 200-1000 mg DSBx2NmG and 0.5-10.0 mg thymosin α-1. Thymosin α-1(Zadaxin or acetylated polypeptide with the following sequence: Ac-Ser-Asp-Ala-Ala-Val-Asp-Thr-Ser-Ser-Glu-Ile-Thr-Thr-Lys-Asp-Leu-Lys-Glu-Lys-Lys-Glu-Val-Val-Glu-Glu-Ala-Glu-Asn-OH) is manufactured by SciClone Pharmaceuticals.

In one embodiment the combination comprises DSB and a tumor necrosis factor. In some embodiments the combination comprises 200-1000 mg DSBx2NmG and tumor necrosis factor-α (TNFα, cachexin or cachectin).

In one embodiment the combination comprises 200-1000 mg DSBx2NmG and 50 μg/m2-200 μg/m2 γ-interferon.

In one embodiment the combination comprises 200-1000 mg DSBx2NmG and 250 μg/m²-500 μg/m² interleukin-3.

In one embodiment the combination comprises 200-1000 mg DSBx2NmG and 0.5 μg/kg-3.0 μg/kg interleukin-4.

One embodiment of the present invention provides a combination comprising an HIV maturation inhibitor and a gene therapy treatment in amounts effective for treatment of HIV when used in a combination therapy. In some embodiments the maturation inhibitor is DSB. In some embodiments, the gene therapy treatment is selected from the group consisting of HGTV-43, M87o, RRz2, and VRX496.

One embodiment of the present invention provides a combination comprising an HIV maturation inhibitor and a cytokine in amounts effective for treatment of HIV when used in a combination therapy. In some embodiments the maturation inhibitor is DSB. In some embodiments, the cytokine is selected from the group consisting of Interferon, interleukin-2, Multikine, and TNF-α. In some embodiments the interferon is Ampligen. In some embodiments the interleukin-2 is BAY 50-4798.

One embodiment of the present invention provides a combination comprising an HIV maturation inhibitor and an assembly inhibitor. In some embodiments the maturation inhibitor is DSB. In some embodiments, the assembly inhibitor is the capsid assembly inhibitor CAI. CAI is developed by Achillion Pharmaceuticals.

One embodiment of the present invention provides a combination comprising an HIV maturation inhibitor and a serotinin re-uptake inhibitor in amounts effective for treatment of HIV when used in a combination therapy. It is believed that a combination of a maturation inhibitor and a SSRI will lead to increases in CD4 baseline counts, the proliferative capacity of peripheral lymphocytes, and decreases in HIV RNA replication. In some embodiments the maturation inhibitor is DSB. In some embodiments, the serotinin re-uptake inhibitor is buspirone. In one embodiment the combination comprises 200-1000 mg DSBx2NmG and 10-75 mg buspirone hydrochloride. Buspirone hydrochloride (Buspar or 8-[4-[4-(2-pyrimidinyl)-1-piperazinyl]butyl]-8-aza-spiro[4.5]decane-7,9-dione monohydrochloride) is manufactured by Bristol-Myers Squibb Company.

One embodiment of the present invention provides a combination comprising an HIV maturation inhibitor and a TAT inhibitor in amounts effective for treatment of HIV when used in a combination therapy. In some embodiments the maturation inhibitor is DSB. In some embodiments, the TAT inhibitor is selected from the group consisting of CGP-64222, RO-247429 and BI-201.

In one embodiment the combination comprises 200-1000 mg DSBx2NmG and RO-247429. RO-247429 is manufactured by Hoffmann-La Roche Pharmaceuticals.

In one embodiment the combination comprises 200-1000 mg DSBx2NmG and BI-201. BI-201 is manufactured by BioInvent.

In one embodiment the combination comprises 200-1000 mg DSBx2NmG and CGP-64222. CGP-64222 is manufactured by Novartis.

One embodiment of the present invention provides a combination comprising an HIV maturation inhibitor and a HIV integrase inhibitor in amounts effective for treatment of HIV when used in a combination therapy. In some embodiments the maturation inhibitor is DSB. In some embodiments, the HIV integrase inhibitor is selected from the group consisting of MK-0518, GS-9137, FZ41, S-1360, L-870812, L-870810, Zintevir (AR-177 or DRG-0297), L731988, L708906, L731927, and L731942. In some embodiments, the HIV integrase inhibitor is a diketo acid, for example, S-1360. In some embodiments, the HIV integrase inhibitor is a napthyridine carboxamide, for example, L-870,812 or L-870,810.

In one embodiment the combination comprises 200-1000 mg DSBx2NmG and 500-2500 mg S-1360. S-1360 is manufactured by Shionogi & Co.

In one embodiment the combination comprises 200-1000 mg DSBx2NmG and MK-0518. MK-0518 is manufactured by Merck.

In one embodiment the combination comprises 200-1000 mg DSBx2NmG and GS-9137. GS-9137 is manufactured by Gilead Sciences.

There is now provided a combination comprising a maturation inhibitor and a secondary pharmaceutical agent selected from the group consisting of antifungals, antibacterials, anti-neoplastics, anti-protozoals, ceragenins, DNA polymerase inhibitors, DNA synthesis inhibitors, anti-HIV antibodies, HIV antisense drugs, IL-2 agonists, α-glucosidase inhibitors, purine nucleoside phosphorylase inhibitors, apoptosis agonists, apoptosis inhibitors, and cholinesterase inhibitors in amounts effective for treatment of HIV when used in a combination therapy.

One embodiment of the present invention provides a combination comprising an HIV maturation inhibitor and an antifungal in amounts effective for treatment of HIV when used in a combination therapy. In some embodiments the maturation inhibitor is DSB. In some embodiments, the antifungal is selected from the group consisting of amphotericin B, clotrimazole, flucytosine, fluconazole, itraconazole, ketoconazole, noxafil, and nystatin.

In one embodiment the combination comprises 200-1000 mg DSBx2NmG and 5-30 mg amphotericin B. Amphotericin B (AMB or Fungizone or [1R-(1R*,3S*,5R*, 6R*,9R*,11R*,15S*,16R*,17R*,18S*,19E,21E,23E,25E,27E,29E,31E,33R*,35S*,36R*,7S*)]-33-[(3-amino-3,6-dideoxy-β-D-mannopyranosyl)-oxy],3,5,6,9,11,17, 37-octahydroxy-15,16,18-trimethyl 13-oxo-14,39-dioxabicyclo[33.3.1]nonatriaconta-19,21,23,25,27,29,31-heptaene-36-carboxylic acid) BMS378806 is manufactured by Bristol-Myers Squibb Company.

In one embodiment the combination comprises 200-1000 mg DSBx2NmG and 5-30 mg clotrimazole. Clotrimazole (Mycelex or 1-(o-chloro-a,a-diphenylbenzyl)imidazole) is manufactured by Bayer.

In one embodiment the combination comprises 200-1000 mg DSBx2NmG and flucytosine. Flucytosine is manufactured by Ancobon.

In one embodiment the combination comprises 200-1000 mg DSBx2NmG and 100-450 mg fluconazole. Fluconazole (Diflucan or 2,4-difluoro-a,a1-bis(1H-1,2,4-triazol-1-ylmethyl)benzyl alcohol) is manufactured by Pfizer.

In one embodiment the combination comprises 200-1000 mg DSBx2NmG and 100-500 mg itraconazole. Itraconazole (Sporanox or (±)-1-[(R*)-sec-butyl]-4-[p-[4-[p-[[(2R*,4S*)-2-(2,4-dichlorophenyl)-2-(1H-1,2,4-triazol-1-ylmethyl)-1,3-dioxolan-4-yl]methoxy]phenyl]-1-piperazinyl]phenyl]-delta2-1,2,4-triazolin-5-one mixture with (±)-1-[(R*)-sec-butyl]-4-[p-[4-[p-[[(2S*4R*)-2-(2,4,-dichlorophenyl)-2-(1H-1,2,4-triazol-1-ylmethyl)-1,3-dioxolan-4-yl]methoxy]phenyl]-1-piperazinyl]phenyl]-delta2-1,2,4-triazolin-5-one or (±)-1-[(RS)-sec-butyl]-4-[p-[4-[p-[[(2R,4S)-2-(2,4-dichlorophenyl)-2-(1H-1,2,4-triazol-1-ylmethyl)-1,3,-dioxolan-4-yl]methoxy]phenyl]-1-piperazinyl]phenyl]-delta2-1,2,4-triazolin-5-one) is manufactured by Janssen-Cilag.

In one embodiment the combination comprises 200-1000 mg DSBx2NmG and 1200-1000 mg ketoconazole. Ketoconazole (Nizoral or cis-1-acetyl-4-[4-[[2-(2,4-dichlorophenyl)-2-(1H-imidazol-1-ylmethyl)-1,3-dioxolan-4-yl]methoxyl]phenyl]piperazine) is manufactured by McNeil Consumer & Specialty Pharmaceuticals.

In one embodiment the combination comprises 200-1000 mg DSBx2NmG and 2-10 mL nystatin. Nystatin (Nilstat or micostatin) is manufactured by Wyeth Pharmaceuticals.

In one embodiment the combination comprises 200-1000 mg DSBx2NmG and noxafil. Noxafil (posaconazole) is manufactured by Schering-Plough.

One embodiment of the present invention provides a combination comprising an HIV maturation inhibitor and an antibacterial in amounts effective for treatment of HIV when used in a combination therapy. In some embodiments the maturation inhibitor is DSB. In some embodiments, the antibacterial is selected from the group consisting of amikacin sulfate, azithromycin, ciprofloxacin, tosufloxacin, clarithromycin, ethambutol, isoniazid, pyrazinamide, rifabutin, rifampin, streptomycin and TLC G-65.

In one embodiment the combination comprises 200-1000 mg DSBx2NmG and 100-1500 mg amikacin sulfate. Amikacin sulfate (O-3-amino-3-deoxy-alpha-D-glucopyranosyl-(1-6)-O-(6-amino-6-deoxy-alpha-D-glucopyranosyl-(1-4))-N1-(4-amino-2-hydroxy-1-oxobutyl)-2-deoxy-D-Streptamine(S)-sulfate (1:2)) is manufactured by Bristol-Myers Squibb Company.

In one embodiment the combination comprises 200-1000 mg DSBx2NmG and azithromycin. Azithromycin (Zmax or zithromax or (2R,3S,4R,5R,8R,10R,11R,12S,13S, 14R)-13-[(2,6-Dideoxy-3-C-methyl-3-O-methyl-α-L-ribo-hexopyranosyl)oxy]-2-ethyl-3,4,10-trihydroxy-3,5,6,8,10,12,14-heptamethyl-11-[[3,4,6-trideoxy-3-(dimethylamino)-β-D-xylo-hexopyranosyl]oxy]-1-oxa-6-azacyclopentadecan-15-one) is manufactured by Pfizer.

In one embodiment the combination comprises 200-1000 mg DSBx2NmG and ciprofloxacin. Ciprofloxacin (Cipro or 1-cyclopropyl-6-fluoro-1,4-dihydro-4-oxo-7-(1-piperazinyl)-3-quinolinecarboxylic acid monohydrate salt) is manufactured by Bayer Corporation.

In one embodiment the combination comprises 200-1000 mg DSBx2NmG and tosufloxacin. Tosufloxacin is manufactured by Abbott Laboratories.

In one embodiment the combination comprises 200-1000 mg DSBx2NmG and 100-500 mg clarithromycin. Clarithromycin (Biaxin or 6-O-methylerythromycin) is manufactured by Abbott Laboratories.

In one embodiment the combination comprises 200-1000 mg DSBx2NmG and 50-400 mg ethambutol. Ethambutol (Myambutol or (+)-2,2′(Ethylenediimino)-di-1-butanol dihydrochloride) is manufactured by X-Gen Pharmaceuticals.

In one embodiment the combination comprises 200-1000 mg DSBx2NmG and 200-1000 mg isoniazid. Isoniazid (Nydrazid) is manufactured by Bristol-Myers Squibb Company.

In one embodiment the combination comprises 200-1000 mg DSBx2NmG and 750-3000 mg pyrazinamide. Pyrazinamide (Zinamide) is manufactured by Merck & Co.

In one embodiment the combination comprises 200-1000 mg DSBx2NmG and rifabutin. Rifabutin (Mycobutin or 1′,4-didehydro-1-deoxy-1,4-dihydro-5′-(2-methylpropyl)-1-oxorifamycin XIV or (9S,12E,14S,15R,16 S,17R,18R,19R, 20S,21S,22E,24Z)-6,16,18,20-tetrahydroxy-1′-isobutyl-14-methoxy-7,9,15,17,19,21,25-hepatmethyl-spiro[9,4-(epoxypentadecal[1,11,13]trienimino)-2H-furo[2′,3′:7,8]naphth[1,2-d]imidazole-2,4′-piperidine]-5,10,26-(3H,9H)-trione-16-acetate) is manufactured by Pfizer.

In one embodiment the combination comprises 200-1000 mg DSBx2NmG and 500-1500 mg rifampin. Rifampin (Rifadin or Rimactane or 3-[[(4-Methyl-1-piperazinyl)imino]methyl]rifamycin or 5,6,9,17,19,21-hexahydroxy-23-methoxy-2,4,12,16,20,22-heptamethyl-8-[N-(4-methyl-1-piperazinyl)formimidoyl]-2,7-(epoxypentadeca[1,11,13]trienimino)naphtho[2,1-b]furan-1,11(2H)-dione21-acetate) is manufactured by MP Biomedicals.

In one embodiment the combination comprises 200-1000 mg DSBx2NmG and streptomycin sulfate. Streptomycin sulfate (O-2-deoxy-2-(methylamino)-(alpha)-L-glucopyranosyl-(1->2)-O-5-deoxy-3-C-formyl-(alpha)-L-lyxofuranosyl-(1->4)-N,N′-bis (aminoiminomethyl)-D-streptamine sulfate (2:3)) is manufactured by Pfizer.

In one embodiment the combination comprises 200-1000 mg DSBx2NmG and gentamicin. Gentamicin (liposomal gentamicin or TLC G-65) is manufactured by Allergan.

One embodiment of the present invention provides a combination comprising an HIV maturation inhibitor and an anti-neoplastic in amounts effective for treatment of HIV when used in a combination therapy. In some embodiments the maturation inhibitor is DSB. In some embodiments, the anti-neoplastic is selected from the group consisting of bevacizumab, halofuginone, metastat, rituximab, COMP (cyclophosphamide, vincristine, methotrexate and prednisone), etoposide, mBACOD (methotrexate, bleomycin, doxorubicin, cyclophosphamide, vincristine and dexamethasone), PRO-MACE/MOPP (prednisone, methotrexate (w/leucovin rescue), doxorubicin, cyclophosphamide, taxol, etoposide/mechlorethamine, vincristine, prednisone and procarbazine), vincristine, vinblastine, angioinhibins, pentosan polysulfate, platelet factor 4 and SP-PG.

In one embodiment the combination comprises 200-1000 mg DSBx2NmG and bevacizumab wherein the bevacizumab is administered at a dose of 2-10 mg/kg as an IV infusion. Bevacizumab (Avastin) is manufactured by Genentech.

In one embodiment the combination comprises 200-1000 mg DSBx2NmG and halofuginone. Halofuginone is manufactured by Collgard Biopharmaceuticals.

In one embodiment the combination comprises 200-1000 mg DSBx2NmG and metastat. Metastat is manufactured by CollaGenex Pharmaceuticals.

In one embodiment the combination comprises 200-1000 mg DSBx2NmG and rituximab wherein the rituximab is administered intravenously once weekly at a dose of 200-500 mg/m2. Rituximab (Rituxan) is manufactured by Genentech.

In one embodiment the combination comprises 200-1000 mg DSBx2NmG and COMP (cyclophosphamide, vincristine, methotrexate and prednisone.

In one embodiment the combination comprises 200-1000 mg DSBx2NmG and etoposide. Etoposide (Etophos or VePesid or VP-16 or 4′-demethylepipodophyllotoxin9-[4,6-0-(R)-ethylidene-(beta)-D-glucopyranoside]) is manufactured by Bristol-Myers Squibb Company.

In one embodiment the combination comprises 200-1000 mg DSBx2NmG and mBACOD (methotrexate, bleomycin, doxorubicin, cyclophosphamide, vincristine and dexamethasone).

In one embodiment the combination comprises 200-1000 mg DSBx2NmG and PRO-MACE/MOPP (prednisone, methotrexate (w/leucovin rescue), doxorubicin, cyclophosphamide, taxol, etoposide/mechlorethamine, vincristine, prednisone and procarbazine).

In one embodiment the combination comprises 200-1000 mg DSBx2NmG and vincristine. Vincristine (leurocristine or LCR or VCR) is manufactured by Index Pharmaceuticals.

In one embodiment the combination comprises 200-1000 mg DSBx2NmG and vinblastine. Vinblastine (Velban or velsar or velbe) is manufactured by Eli Lilly & Co.

In one embodiment the combination comprises 200-1000 mg DSBx2NmG and an angioinhibin, for example, Fumagillin. Fumagillin is manufactured by Triangle Pharmaceuticals.

In one embodiment the combination comprises 200-1000 mg DSBx2NmG and Elmiron. Elmiron(pentosan polysulfate) is manufactured by Ortho-McNeil.

In one embodiment the combination comprises 200-1000 mg DSBx2NmG and platelet factor 4. Platelet factor 4 is manufactured by Haematologic Technologies.

In one embodiment the combination comprises 200-1000 mg DSBx2NmG and SP-PG. SP-PG is manufactured by Daiichi Pharmaceuticals.

One embodiment of the present invention provides a combination comprising an HIV maturation inhibitor and an anti-protozoal in amounts effective for treatment of HIV when used in a combination therapy. In some embodiments the maturation inhibitor is DSB. In some embodiments, the anti-protozoal is selected from the group consisting of albendazole, azithromycin, clarithromycin, clindamycin, corticosteroids, dapsone, DIMP, eflornithine, 566C80, fansidar, furazolidone, L-671329, letrazuril, metronidazole, paromycin, pefloxacin, pentamidine, piritrexim, primaquine, pyrimethamine, somatostatin, spiramycin, sulfadiazine, trimethoprim, TMP/SMX, trimetrexate and WR 6026.

One embodiment of the present invention provides a combination comprising an HIV maturation inhibitor and a DNA polymerase inhibitor or DNA synthesis inhibitor in amounts effective for treatment of HIV when used in a combination therapy. In some embodiments the maturation inhibitor is DSB. In some embodiments, the DNA polymerase inhibitor or DNA synthesis inhibitor is selected from the group consisting of Foscavir, ACH-126443 (L-2′,3′-didehydro-dideoxy-5-fluorocytidine), entecavir ((1S,3S,4S)-9-[4-hydroxy-3-(hydroxymethyl)-2-methylenecyclopent-yl]guanine), calanolide A ([10R-(10α,11β,12α)]-11,12-dihy-dro-12-hydroxy-6,6,10,11-tetramethyl-4-propyl-2H,6H,10H-benzo[1,2-b:3,4-b′-:5,6-b″]tripyran-2-one), calanolide B, NSC-674447 (1,1′azobisformamide), Iscador (viscum alubm extract), and Rubetecan.

One embodiment of the present invention provides a combination comprising an HIV maturation inhibitor and an antisense drug in amounts effective for treatment of HIV when used in a combination therapy. In some embodiments the maturation inhibitor is DSB. In some embodiments, the antisense drug is selected from the group consisting of HGTV-43, and GEM-92. HGTV-43 (Human Gene Transfer Vector-43) is manufactured by Enzo Pharmaceuticals.

One embodiment of the present invention provides a combination comprising an HIV maturation inhibitor and a ceragenin in amounts effective for treatment of HIV when used in a combination therapy. In some embodiments the maturation inhibitor is DSB. In some embodiments, the ceragenin is CSA-54. In one embodiment the combination comprises 200-1000 mg DSBx2NmG and CSA-54. CSA-43 is manufactured by Ceragenix Pharmaceuticals.

One embodiment of the present invention provides a combination comprising an HIV maturation inhibitor and an anti-HIV antibody in amounts effective for treatment of HIV when used in a combination therapy. In some embodiments the maturation inhibitor is DSB. In some embodiments, the anti-HIV antibody is selected from the group consisting of NM-01, PRO-367, KD-247, Cytolin, TNX-355 (CD4 antibody), AGT-1, PRO-140, or anti-CTLA-4 Mab.

One embodiment of the present invention provides a combination comprising an HIV maturation inhibitor and an HIV vaccine in amounts effective for treatment of HIV when used in a combination therapy. In some embodiments the maturation inhibitor is DSB. In some embodiments, the HIV vaccine is selected from the group consisting of Alvac, Aidsvax, Remune, HIV gp41 vaccine, HIV gp120 vaccine, HIV gp160 vaccine, HIV p17 vaccine, HIV p24 vaccine, HIV p55 vaccine, AlphaVax Vector System, canarypox gp160 vaccine, AntiTat, MVA-F6 Nef vaccine, HIV rev vaccine, C4-V3 peptide, p2249f, VIR-201, HGP-30W, TBC-3B, and PARTICLE-3B.

One embodiment of the present invention provides a combination comprising an HIV maturation inhibitor and an interferon or interferon agonist in amounts effective for treatment of HIV when used in a combination therapy. In some embodiments the maturation inhibitor is DSB. In some embodiments, the interferon or interferon agonist is selected from the group consisting of Sumiferon, MultiFeron, interferon-τ, Reticulose, Intron-A, and Human leukocyte interferon-α.

One embodiment of the present invention provides a combination comprising an HIV maturation inhibitor and a TNF-α antagonist in amounts effective for treatment of HIV when used in a combination therapy. In some embodiments the maturation inhibitor is DSB. In some embodiments, the TNF-α antagonist is selected from the group consisting of thalidomide, infliximab, and curdlan sulfate.

One embodiment of the present invention provides a combination comprising an HIV maturation inhibitor and an α-glucosidase inhibitor in amounts effective for treatment of HIV when used in a combination therapy. In some embodiments the maturation inhibitor is DSB. In some embodiments, the α-glucosidase inhibitor is Bucast.

One embodiment of the present invention provides a combination comprising an HIV maturation inhibitor and a purine nucleoside phosphorylase inhibitor in amounts effective for treatment of HIV when used in a combination therapy. In some embodiments the maturation inhibitor is DSB. In some embodiments, the purine nucleoside phosphorylase inhibitor is peldesine (2-amino-4-oxo-3H,5H-7-[(3-pyridyl)methyl]pyrrolo[3,2-d]pyrimidine).

One embodiment of the present invention provides a combination comprising an HIV maturation inhibitor and an apoptosis agonist or inhibitor in amounts effective for treatment of HIV when used in a combination therapy. In some embodiments the maturation inhibitor is DSB. In some embodiments, the apoptosis agonist or inhibitor is selected from the group consisting of Arkin Z, Panavir, or Coenzyme Q10.

One embodiment of the present invention provides a combination comprising an HIV maturation inhibitor and a cholinesterase inhibitor in amounts effective for treatment of HIV when used in a combination therapy. In some embodiments the maturation inhibitor is DSB. In some embodiments, the cholinesterase inhibitor is Cognex.

One embodiment of the present invention provides a combination comprising an HIV maturation inhibitor and an anti-viral agent in amounts effective for treatment of HIV when used in a combination therapy. In some embodiments the maturation inhibitor is DSB. In some embodiments, the anti-viral agent is selected from the group consisting of ribavirin, cellcept, amantadine, merimepodib, ANA975, CPG 10101, IDN-6556, BILN-2061, VRX-950, SCH6, SCH7, ACH-806, PSI-6130, Viramidine, Levovorin, valopicitabine, merimepodib, R803, HCV-086, HCV-796, JTK-003, JTK-109, NV-08, HCV-SM, R1626, ISIS014803, Hepex-C, HCV-AB68, Cicavir, HuMax-HepC.]

In one embodiment the combination comprises 200-1000 mg DSBx2NmG and ribavirin. Ribavirin (1-β-D-ribofuranosyl-1H-1,2,4-triazole-3-carboxamide) is manufactured by Roche and Valeant Pharmaceuticals.

In one embodiment the combination comprises 200-1000 mg DSBx2NmG and 200-1000 mg cellcept wherein the cellcept may be administered orally or intravenously. Cellcept (mycophenolate mofetil or MMF or 2-morpholinoethyl(E)-6-(1,3-dihydro-4-hydroxy-6-methoxy-7-methyl-3-oxo-5-isobenzofuranyl)-4-methyl-4-hexenoate) is manufactured by Hoffmann-La Roche Inc.

In one embodiment the combination comprises 200-1000 mg DSBx2NmG and 100-300 mg amantadine. Amantadine (Symmetrel or 1-adamantanamine hydrochloride) is manufactured by Novartis.

In one embodiment the combination comprises 200-1000 mg DSBx2NmG and 100-1000 mg merimepodib. Merimepodib (VX-947) is manufactured by Vertex Pharmaceuticals.

In one embodiment the combination comprises 200-1000 mg DSBx2NmG and 300-1500 mg ANA975. ANA975 is manufactured by Anadys Pharmaceuticals.

In one embodiment the combination comprises 200-1000 mg DSBx2NmG and 0.25-20 mg Actilon wherein the Actilon may be delivered subcutaneously. Actilon (CPG 10101) is manufactured by Coley Pharmaceutical Group.

In one embodiment the combination comprises 200-1000 mg DSBx2NmG and 40-200 mg IDN-6556. IDN-6556 (3-{2-(2-tert-butyl-phenylaminooxalyl)-amino]-propionylamino}-4-oxo-5-(2,3,5,6-tetrafluoro-phenoxy)-pentanoic acid) is manufactured by Idun Pharmaceuticals.

In one embodiment the combination comprises 200-1000 mg DSBx2NmG and 150-2000 mg BILN-2061. BILN-2061 is manufactured by Boehringer Ingelheim.

In one embodiment the combination comprises 200-1000 mg DSBx2NmG and VR-950. VR-950 is manufactured by Vertex Pharmaceuticals.

In one embodiment the combination comprises 200-1000 mg DSBx2NmG and SCH6. SCH6 is manufactured by Schering Plough Corp.

In one embodiment the combination comprises 200-1000 mg DSBx2NmG and SCH7. SCH7 is manufactured by Schering Plough Corp.

In one embodiment the combination comprises 200-1000 mg DSBx2NmG and ACH-806. ACH-806 (GS 9132) is manufactured by Achillion Pharmaceuticals.

In one embodiment the combination comprises 200-1000 mg DSBx2NmG and PSI-6130. PSI-6130 (R1656) is manufactured by Pharmasset and Hoffmann-La Roche Inc.

In one embodiment the combination comprises 200-1000 mg DSBx2NmG and 300-1200 mg Viramidine. Viramidine (1-β-D-ribofuranosyl-1H-1,2,4-triazole-3-carboximide) is manufactured by Valeant Pharmaceuticals.

In one embodiment the combination comprises 200-1000 mg DSBx2NmG and Levovorin. Levovorin (LVV or L-isomer of ribavirin) is manufactured by Valeant Pharmaceuticals.

In one embodiment the combination comprises 200-1000 mg DSBx2NmG and 200-1000 mg valopicitabine. Valopicitabine (va1-LdC or LdC or 3′-O-(L-valyl)-2′-C-methylcytidine or 4-amino-1-[3-O-[(2S)-2-amino-3-méthylbutanoyl]-2-C-méthyl-β-D-ribofuranosyl]pyrimidin-2(1H)-one) is manufactured by Idenix and Novartis.

In one embodiment the combination comprises 200-1000 mg DSBx2NmG and R803. R803 is manufactured by Rigel.

In one embodiment the combination comprises 200-1000 mg DSBx2NmG and HCV-086 is manufactured by Wyeth and Viropharma.

In one embodiment the combination comprises 200-1000 mg DSBx2NmG and HCV-796. HCV-796 is manufactured by Wyeth and Viropharma.

In one embodiment the combination comprises 200-1000 mg DSBx2NmG and JTK-003. JTK-003 is manufactured by Japan Tobacco and Akros.

In one embodiment the combination comprises 200-1000 mg DSBx2NmG and JTK-109. JTK-109 is manufactured by Japan Tobacco and Akros.

In one embodiment the combination comprises 200-1000 mg DSBx2NmG and NV-08. NV-08 is manufactured by Idenix and Novartis.

In one embodiment the combination comprises 200-1000 mg DSBx2NmG and HCV-SM. HCV-SM is manufactured by XTL Biopharmaceuticals.

In one embodiment the combination comprises 200-1000 mg DSBx2NmG and R1626. R1626 is manufactured by Hoffmann-La Roche Inc.

In one embodiment the combination comprises 200-1000 mg DSBx2NmG and ISIS014803. ISIS014803 is manufactured by Isis.

In one embodiment the combination comprises 200-1000 mg DSBx2NmG and Hepex-C. Hepex-C is manufactured by XTL Biopharmaceuticals.

In one embodiment the combination comprises 200-1000 mg DSBx2NmG and HCV-AB68. HCV-AB68 is manufactured by XTL Biopharmaceuticals.

In one embodiment the combination comprises 200-1000 mg DSBx2NmG and Cicavir. Cicavir is manufactured by Nabi Biopharmaceuticals.

In one embodiment the combination comprises 200-1000 mg DSBx2NmG and HuMax-HepC. HuMax-HepC is manufactured by Genmab A/S.

One embodiment of the present invention provides a combination comprising an HIV maturation inhibitor and a secondary pharmaceutical agent in amounts effective for treatment of HIV when used in a combination therapy. In some embodiments the maturation inhibitor is DSB. In some embodiments, the anti-viral is selected from the group consisting of adefovir, adefovir dipivoxil, clevudine, telbivudine, Alamifovir, Remofovir, LB80380, NOV-205, EHT-899, HepeX-B, SpecifEx-HepB, LY-582563, EHT-899, CellExSys, and NOV-2053.

In one embodiment the combination comprises 200-1000 mg DSBx2NmG and adefovir. Adefovir (Preveon) is manufactured by Gilead Sciences.

In one embodiment the combination comprises 200-1000 mg DSBx2NmG and 0.05 mg to 1 mg entecavir. Entecavir (Baraclude) is manufactured by Bristol Meyers Squibb.

In one embodiment the combination comprises 200-1000 mg DSBx2NmG and adefovir dipivoxil. Adefovir dipivoxil (Hepsera or 9-[2-[bis[(pivaloyloxy)methoxy]phosphinyl]methoxy]-ethyl]adenine) is manufactured by Gilead Sciences.

In one embodiment the combination comprises 200-1000 mg DSBx2NmG and 5-75 mg clevudine. Clevudine (L-FMAU) is manufactured by Pharmasset.

In one embodiment the combination comprises 200-1000 mg DSBx2NmG and 200-1000 mg telbivudine. Telbivudine (LdT) is manufactured by Idenix Pharmaceuticals.

In one embodiment the combination comprises 200-1000 mg DSBx2NmG and 20-100 mg LB80380. LB80380 (ANA380) is manufactured by LG Sciences.

In one embodiment the combination comprises 200-1000 mg DSBx2NmG and 1-250 mg Alamifovir. Alamifovir ([[2-[2-amino-6-[(4-méthoxyphenyl)sulfanyl]-9H-purin-9-yl]éthoxy]méthyl]phosphonate de bis(2,2,2-trifluoroéthyle) is manufactured by Eli Lilly & Co.

In one embodiment the combination comprises 200-1000 mg DSBx2NmG and 2-100 mg Remofovir. Remofovir (CS-917, pradefovir mesylate) is manufactured by Valeant.

In one embodiment the combination comprises 200-1000 mg DSBx2NmG and 10-75 mg Molixan. Molixan (NOV-205 or bis-glycine-L-cysteinyl-bis-(g-L-glutamate).9-b-D-ribofuranozilhypoxanthine) is manufactured by Novelos.

In one embodiment the combination comprises 200-1000 mg DSBx2NmG and EHT-899 is manufactured by Enzo Biochem.

In one embodiment the combination comprises 200-1000 mg DSBx2NmG and 10-80 mg HepeX-B wherein the HepeX-B may be delivered parenterally. HepeX-B is manufactured by Cubist Pharmaceuticals.

In one embodiment the combination comprises 200-1000 mg DSBx2NmG and SpecifEx-HepB is manufactured by CellExSys.

In one embodiment the combination comprises 200-1000 mg DSBx2NmG and LY-582563. LY-582563 is manufactured by Eli Lilly &Co.

In one embodiment the combination comprises 200-1000 mg DSBx2NmG and NOV-205. NOV-205 (Bam-205) is manufactured by Novelos Therapeutics.

Oral dosages of DSBx2NmG for combination therapy may be any dosage ranging from about 50 mg to about 1000 mg of DSB free acid per administration. For example, single administrations of DSBx2NmG as a component of combination therapy may be 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, or 1000 mg of the free acid of DSB per administration.

In some embodiments, about 200 mg to about 1000 mg of the free acid of DSB is administered multiple times per day in a combination of the present invention. The amount of one or more salts administered per day is determined by the total amount of one or more salts administered in a 24 hour period. Thus, dosage regimens which instruct administration of one or more salts of the invention multiple times during a 24 hour period are within the scope of the invention if the cumulative amount administered during a 24 hour period is within the ranges listed above.

In some embodiments, about 200 mg to about 1000 mg of the free acid of DSB is administered once per day in a combination of the present invention. In preferred embodiments, the combination comprises two components, DSB and a second pharmaceutical agent, where both components are suitable for once a day administration. DSBx2NmG is suitable for once a day administration. Other combination components suitable for once a day administration include: abacavir (two 300 mg tablets), stavudine as present in the Zerit XR formulation marketed as of the filing date of the present application (one 100 mg XR capsule), didanosine as present in the Videx-EC formulation marketed as of the filing date of the present application (one 200 to 400 mg EC capsule), Viread (300 mg tablet), efavirenz (600 mg tablet or three 200 mg capsules), TMC-278 (one 25-150 mg oral dose), emtricitabine (one 200 mg capsule), abacavir and 3TC as present in the Epzicom formulation marketed as of the filing date of the present application (one tablet comprising 600 mg abacavir and 300 mg 3TC), tenofovir and emtricitabine as present in the Truvada formulation marketed as of the filing date of the present application (one tablet comprising 300 mg tenofovir+200 mg emtricitabine), and tenofovir (one 300 mg tablet).

In one embodiment the combination comprises about 200 mg to about 1000 mg of the free acid of DSB in combination with lopinavir and ritonavir as present in the Kaletra formulation marketed as of the filing date of the present application (four or six capsules each comprising 133 mg lopinavir and 33 mg of ritonavir).

In one embodiment the combination comprises about 200 mg to about 1000 mg of the free acid of DSB in combination with abacavir (two 300 mg tablets).

In one embodiment the combination comprises about 200 mg to about 1000 mg of the free acid of DSB in combination with zidovudine (two 600 mg doses).

In one embodiment the combination comprises about 200 mg to about 1000 mg of the free acid of DSB in combination with stavudine as present in the Zerit XR formulation marketed as of the filing date of the present application (one 100 mg XR capsule).

In one embodiment the combination comprises about 200 mg to about 1000 mg of the free acid of DSB in combination with TMC-278 (one 25-150 mg oral dose.

In one embodiment the combination comprises about 200 mg to about 1000 mg of the free acid of DSB in combination with didanosine as present in the Videx-EC formulation marketed as of the filing date of the present application (one 200 to 400 mg EC capsule).

In one embodiment the combination comprises about 200 mg to about 1000 mg of the free acid of DSB in combination with Viread (300 mg tablet).

In one embodiment the combination comprises about 200 mg to about 1000 mg of the free acid of DSB in combination with efavirenz (600 mg tablet or three 200 mg capsules).

In one embodiment the combination comprises about 200 mg to about 1000 mg of the free acid of DSB in combination with lamivudine (two 150 mg tablets or one 300 mg tablet).

In one embodiment the combination comprises about 200 mg to about 1000 mg of the free acid of DSB in combination with nevirapine (two 200 mg tablets).

In one embodiment the combination comprises about 200 mg to about 1000 mg of the free acid of DSB in combination with fosamprenavir and ritonavir as present in the Lexiva formulation marketed as of the filing date of the present application (two 700 mg tablets and two 100 mg ritonavir capsules).

In one embodiment the combination comprises about 200 mg to about 1000 mg of the free acid of DSB in combination with saquinavir (one 1600 mg capsule and one 200 mg ritonavir capsule).

In one embodiment the combination comprises about 200 mg to about 1000 mg of the free acid of DSB in combination with emtricitabine (one 200 mg capsule).

In one embodiment the combination comprises about 200 mg to about 1000 mg of the free acid of DSB in combination with abacavir and 3TC as present in the Epzicom formulation marketed as of the filing date of the present application (one tablet comprising 600 mg abacavir and 300 mg 3TC).

In one embodiment the combination comprises about 200 mg to about 1000 mg of the free acid of DSB in combination with tenofovir and emtricitabine as present in the Truvada formulation marketed as of the filing date of the present application (one tablet comprising 300 mg tenofovir+200 mg emtricitabine).

In one embodiment the combination comprises about 200 mg to about 1000 mg of the free acid of DSB in combination with tenofovir (one 300 mg tablet).

Such combination therapies comprising two or more components where each component is suitable for once a day administration offer benefits including increased flexibility in dosing schedules, increased patient compliance, and improved quality of life for patients.

Some combinations of the present invention include a component comprising at least one salt of DSB. While individual needs vary, determination of optimal ranges of effective amounts of each component is within the skill of the art. Typical therapeutically effective amounts of a DSB salt can comprise about 0.05 to about 100 mg/kg body weight. In some embodiments, a therapeutically effective amount of a DSB salt comprises about 0.1 to about 100 mg/kg body weight of the active ingredient, preferably about 0.1 to about 20 mg/kg body weight of the active ingredient. In some embodiments, a more preferred therapeutically effective amount of a DSB salt comprises about 0.2 to about 10 mg/kg body weight. A therapeutically effective amount of a DSB salt can comprise about 0.5 to about 5 mg/kg body weight. In some embodiments, a therapeutically effective amount of a DSB salt can comprise about 10 to about 100 mg/kg body weight.

Various dosage amounts of the composition of the invention can be administered to provide various plasma levels of DSB. In some embodiments, a preferred dosage amount is one which provides a trough concentration of DSB in the patient's plasma of about 1 micromolar (μM) to about 1 millimolar (mM). In some embodiments, the dosage amount provides a trough concentration of DSB in the patient's plasma of about 4 μM (2.34 μg/mL) to about 1000 μM, about 40 μM to about 1000 μM, or about 400 μM to about 1000 μM. In some embodiments, the dosage amount provides a trough concentration of DSB in the patient's plasma of about 4 μM (2.34 μg/mL) to about 200 μM, about 10 μM to about 200 μM, or about 40 μM to about 200 μM. In some embodiments, the dosage amount provides a trough concentration of DSB in the patient's plasma of at least about 4 μM (2.34 μg/mL) or greater, at least about 10 μM or greater, at least about 40 μM or greater, at least about 100 μM or greater, or at least 200 μM or greater. In some embodiments, the dosage amount provides a trough concentration of DSB in the patient's plasma of about 400 μM. The “trough concentration” is the concentration of DSB in the patient's plasma immediately prior to subsequent dosing of the patient.

### EXAMPLE 1

### Determination of Individual Compound EC50 on MT-2 Cell-Line

Drug Preparation

The EC50 value for each compound is determined prior to determining the combination index. Each compound is tested in replicates of three with a starting concentration of 1 μg/ml. Compounds are dissolved in DMSO at a stock concentration of 10 mg/ml. Test compounds are serially diluted (4-fold dilutions) in a 96-flat-bottom-well plate. The initial working concentration of compound is 4× the desired final concentration. The final volume of drug per well is 45 μl. Included as controls are at least four wells containing cells only and four containing cells and virus in the absence of drug.

Virus Preparation

The HIV-1IIIB virus generated in the H9 cell line is diluted in culture media. The virus is used at a dilution that will cause 50%-75% cell death in the control well on day five post-infection as determined by XTT/PMS vital dye. Virus (45 μl) is added to all wells containing drug as well as the control wells.

Cell Preparation

MT-2 cells are added to each well at a volume of 90 μl per well at a cell concentration of 3.3×10⁵ cells per ml. MT-2 cells are human T-cell leukemia cells isolated from cord blood lymphocytes and co-cultured with cells from patients with adult T-cell leukemia. Transformed with and continuous producer of the HTLV-1 virions. The MT-2 cell line was acquired from the AIDS Research and Reference Reagent Program.

Assay

Culture plates are placed in a 37° C., 5% CO₂, humidified incubator. On day 5, post infection cell viability is determined using XTT/PMS dye (Roehm, et al. 1991). XTT, (2,3-Bis(2-methoxy-4-nitro-5-sulfophenyl)-2H-tetrazolium-5-carboxanilide) is a yellow tetrazolium salt that, in the presence of dehydrogenase enzymes of metabolically active cells, is reduced to yield a soluble orange formazan dye, which can be measured by absorbance at 490 (or 450) nm in a microplate reader. PMS, phenazine methosulfate, is an electron coupling agent that increases the efficiency of the bioreduction of XTT. The use of tetrazolium salts, including XTT and XTT/PMS, in virologic assays is an established practice known to those of skill in the art. XTT/PMS viability dye is prepared fresh as follows: 1.25 mg/ml stock solution of XTT is prepared in culture media pre-warmed to 60° C. and 0.18 mg/ml stock solution of PMS is prepared in PBS. For each plate to be analyzed, 5 ml of XTT/PMS is needed. PMS stock solution (200 μl) is added to 5 ml of stock XTT, then 50 μl of the XTT/PMS mixture is added to each well of the plate. Plates are placed uncovered into a 5% CO₂, 37° C. incubator and read on plate reader at a wavelength of 450 nm after four hour incubation.

Data Compilation

The percent inhibition at a given concentration is determined by using the following formula: (average OD−virus control)/(cell control−virus control). The EC50 for each compound is calculated from the linear regression of percent inhibition. Figure A, below, demonstrates calculation of the EC50 of DSB. Other EC50 calculations were determined according to the same procedure.

[image]

### EXAMPLE 2

### Determination of Co-Action of Two Compound Combinations

Compound Preparation

The starting concentration for each drug is based on the EC50 values determined above. The compounds are tested in triplicates, at a starting concentration that will allow each compound to reach their EC50 value at approximately the same dilution. Compound dilutions are made in a 96-well flat bottom assay plate starting at 8× the desired starting concentration. Compounds are combined at a 1:1 volume ratio. Then seven 4-fold serial dilutions are made, leaving a final volume of 45 μl of the combined drug. Each compound is also tested independently to determine the EC50 for the compound alone (similar to method described in Example 1). Control wells containing virus and cells only and cell only wells are also included. All dilutions are made into tissue culture media (RPMI-1640, 10% FBS, and 50 μg/ml gentamicin). Table 1 summarizes the starting concentrations for each compound.

TABLE 1

| | | | Starting |
|---|---|---|---|
| Class of | Compound | | concentrations |
| Component | Number | Compound | (μg/ml) |
| Maturation | 2.0 | DSB | 0.04 |
| Inhibitor | | | 0.26 |
| NRTI | 2.1 | AZT | 0.32 |
| | 2.2 | Stavudine | 1.00 |
| | 2.3 | Lamivudine | 3.20 |
| | 2.4 | Didanosine | 10.80 |
| | 2.5 | Abacavir | 3.60 |
| | 2.6 | Tenofovir | 14.0 |
| | 2.7 | Emtricibine | 1.2 |
| NNRTI | 2.8 | Nevirapine | 0.10 |
| | 2.9 | Efavirenz | 0.000912 |
| | | | 0.001000 |
| | | | 0.000050 |
| Protease | 2.10 | Indinavir | 0.07 |
| | 2.11 | Nelfinavir | 0.05 |
| | | | 0.13 |
| | | | 1.00 |
| | 2.12 | Saquinavir | 0.10 |
| | 2.13 | Ritonavir | 1.00 |
| | 2.14 | Amprenavir | 2.20 |
| | 2.15 | Lopinavir | 0.37 |
| | 2.16 | Atazanavir | 0.04 |
| Fusion | 2.17 | Enfuvuritde | 10.0 |

Virus and Cell Preparation

An equal volume of H9/HIV-1IIIB virus is added to each well containing drug. The virus is prepared as described above. 90 μl of MT-2 cells (described previously) at a concentration of 3.3×10⁵ cells per ml are added to each well.

Assay

Culture plates are placed in a 37° C., 5% CO₂, humidified incubator for 5 days. On day 5-post infection, plates are visually scored to determine the level of drug inhibition and the cytopathic effect of the virus in all experimental and control wells. Cell viability is then determined using XTT/PMS dye as described above. Briefly, the Vital dye is added to each well and after four hour incubation, the plates are read on a plate reader at the wavelength of 450 nm.

Data Compilation

The percent inhibition at a given single drug combination or co-combination is determined using the following formula: (average OD−virus control)/(cell control−virus control). EC50 is calculated from the linear regression of percent inhibition. Those points which fall in the linear range of the curve are entered into the CalcuSyn software (described below).

Quantification of the co-action exhibited by some combinations of the present invention, data was calculated with CalcuSyn software applying the Chou-Talalay equation of dose-effect relationships of multiple drugs (Chou, T. C. and Talalay P. Quantitative analysis of dose-effect relationships: The combined effects of multiple drugs or enzyme inhibitors. Adv. Enz. Regul. 22:27-55, 1984). The Chou-Talalay equation is:

$$CI = {\sum }_{j = 1}^{n} \frac{{( D )}_{j}}{{( {ED}_{90} )}_{j}}$$

Where ‘D’ is the dose of the drug; “ED90” is the drug concentration that results in a 90% reduction in virus replication; “CI” is the combination index where a combination having a CI of less than or equal to 0.9 is termed synergistic, a combination having a CI between 0.9 and 1.10 is termed additive, and a combination having a CI of equal to or greater than 1.10 is termed antagonistic. “CI90” is the combination index resulting in a 90% reduction in virus replication. Table 2 summarizes the C190 of PA-457 against several classes of HIV-1 inhibitors.

TABLE 2

| | Experiment | |
|---|---|---|
| Combination | Number | CI₉₀ |
| DSB + AZT | 1a | 0.329 |
| | 1b | 0.159 |
| | Average | 0.243 |
| | SD | 0.120 |
| DSB + Stavudine | 2a | 0.258 |
| | 2b | 0.637 |
| | Average | 0.448 |
| | SD | 0.268 |
| DSB + Lamivudine | 3a | 1.173 |
| | 3b | 0.518 |
| | 3c | 0.378 |
| | Average | 0.690 |
| | SD | 0.424 |
| DSB + Didanosine | 4a | 0.293 |
| | 4b | 0.198 |
| | Average | 0.245 |
| | SD | 0.067 |
| DSB + Abacavir | 5a | 0.205 |
| | 5b | 0.539 |
| | Average | 0.372 |
| | SD | 0.237 |
| DSB + Tenofovir | 6a | 0.511 |
| | 6b | 0.753 |
| | Average | 0.632 |
| | SD | 0.171 |
| DSB + Emtricibine | 7a | 0.739 |
| | 7b | 1.052 |
| | Average | 0.895 |
| | SD | 0.222 |
| DSB + Nevarapine | 8a | 0.446 |
| | 8b | 0.603 |
| | Average | 0.524 |
| | SD | 0.111 |
| DSB + Efavirenz | 9a | 0.537 |
| | 9b | 1.719 |
| | 9c | 0.824 |
| | 9d | 0.835 |
| | Average | 0.979 |
| | SD | 0.513 |
| DSB + Indinavir | 11a | 0.631 |
| | 11b | 0.563 |
| | Average | 0.597 |
| | SD | 0.048 |
| DSB + Nelfinavir | 12a | 0.829 |
| | 12b | 0.189 |
| | 12c | 0.192 |
| | 12d | 0.519 |
| | Average | 0.432 |
| | SD | 0.306 |
| DSB + Enfuvirtide | 13a | 1.083 |
| | 13b | 0.980 |
| | Average | 1.031 |
| | SD | 0.073 |
| DSB + Saquinavir | 14a | 1.052 |
| | 14b | 0.774 |
| | 14c | 0.666 |
| | Average | 0.720 |
| | SD | 0.076 |
| DSB + Ritonavir | 15a | 0.266 |
| | 15b | 0.478 |
| | Average | 0.372 |
| | SD | 0.150 |
| DSB + Lopinavir | 16a | 0.492 |
| | 16b | 0.829 |
| | Average | 0.660 |
| | SD | 0.239 |
| DSB + Atazanavir | 17a | 1.016 |
| | 17b | 1.104 |
| | Average | 1.060 |
| | SD | 0.062 |
| DSB + Amprenavir | 18a | 1.417 |
| | 1b8 | 0.708 |
| | Average | 1.062 |
| | SD | 0.502 |

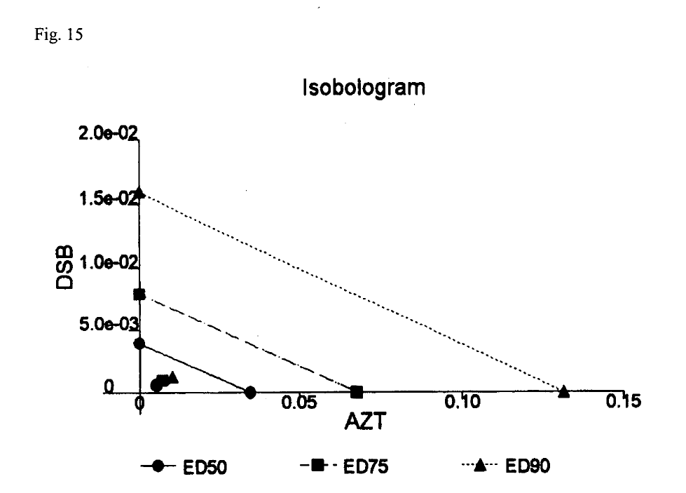

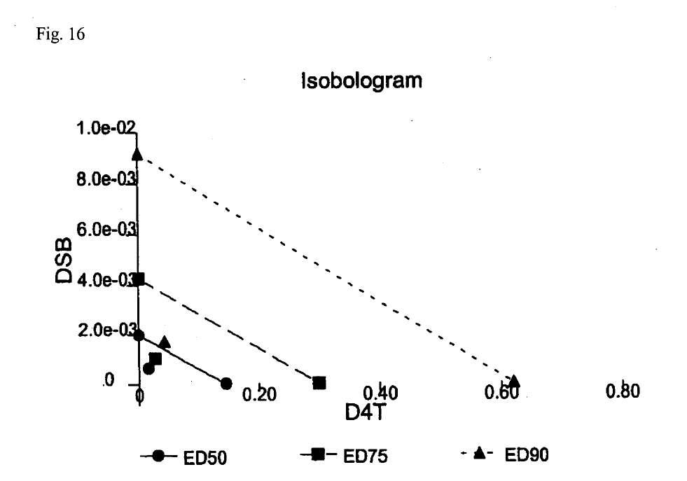

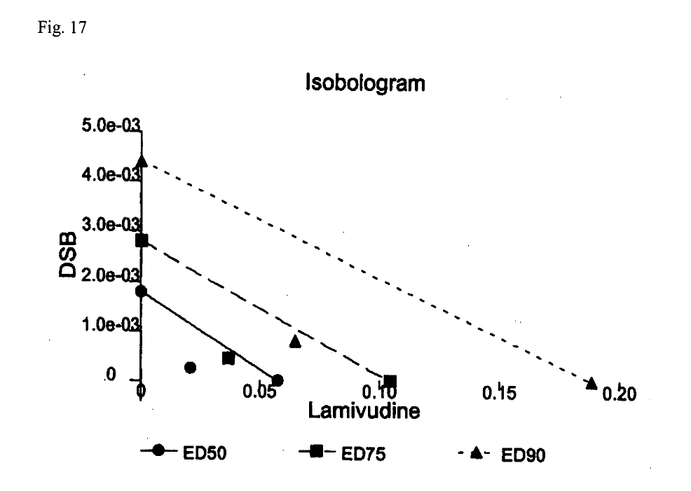

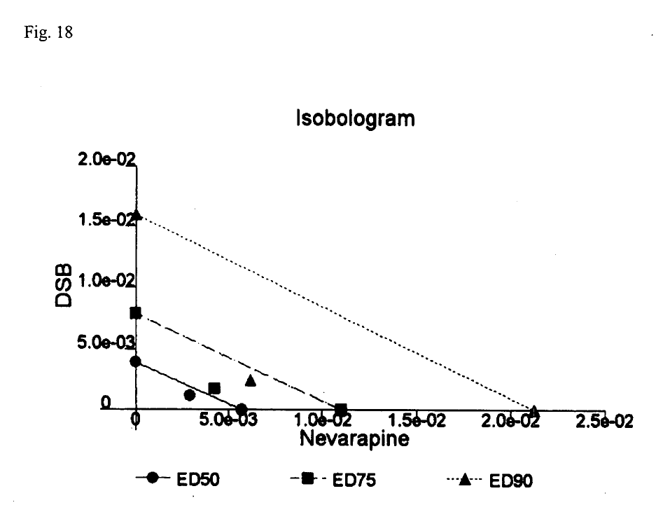

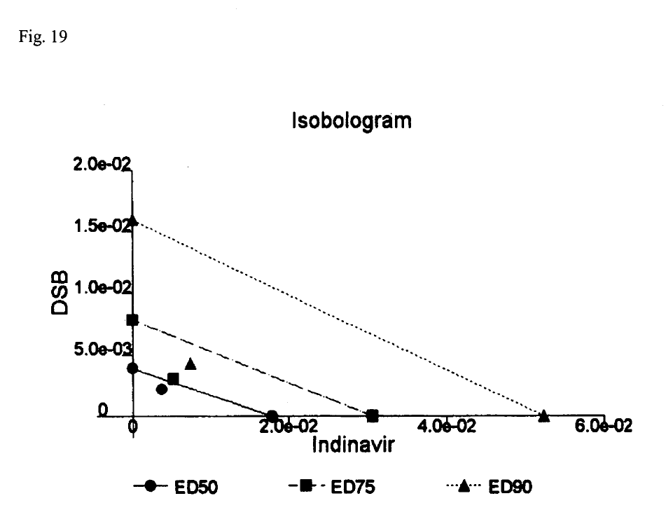

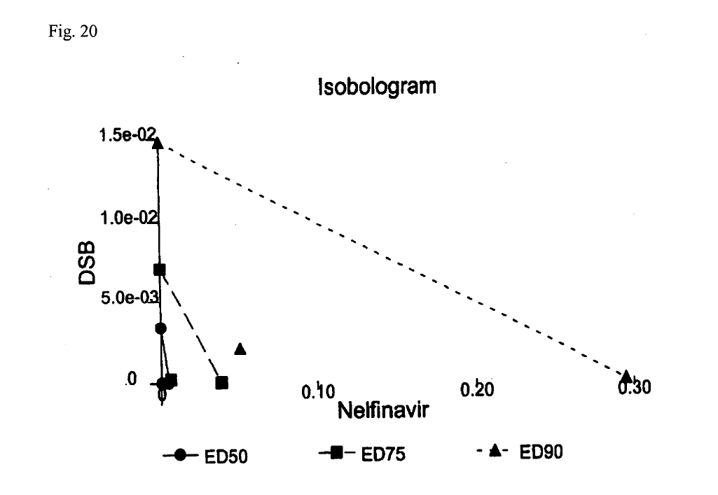

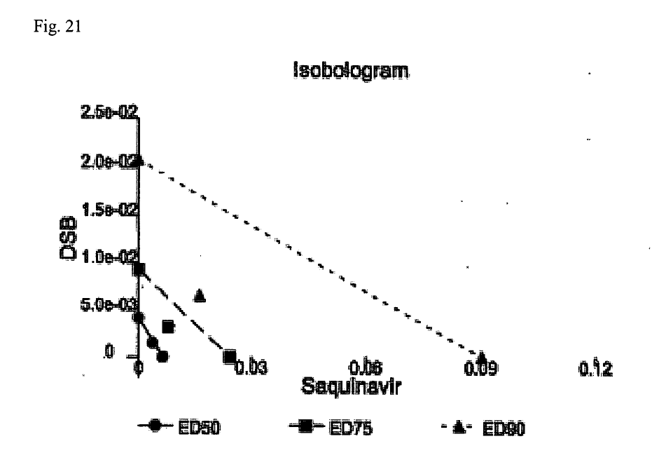

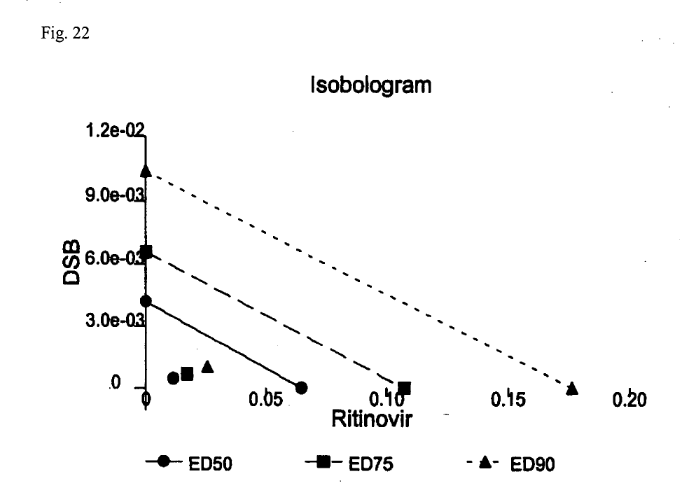

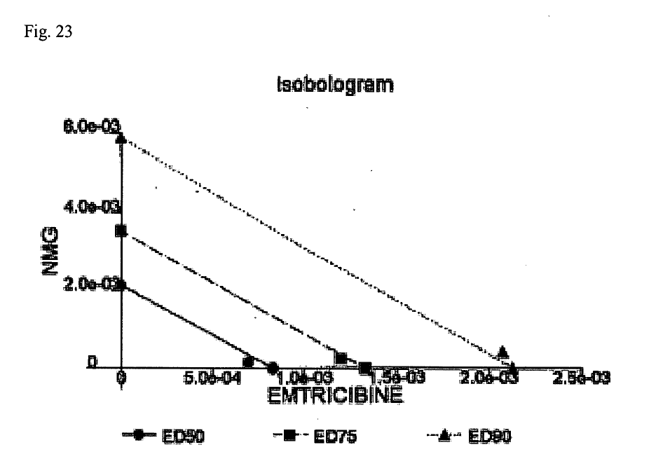

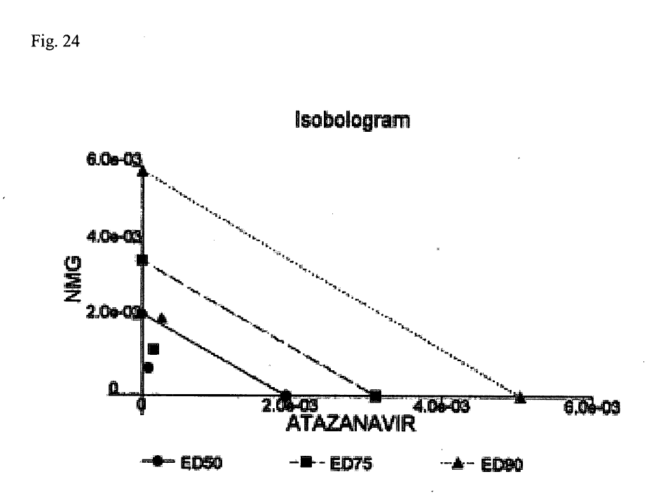

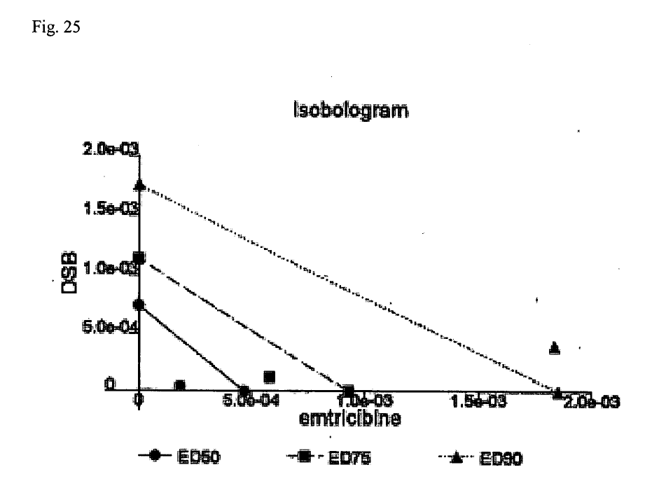

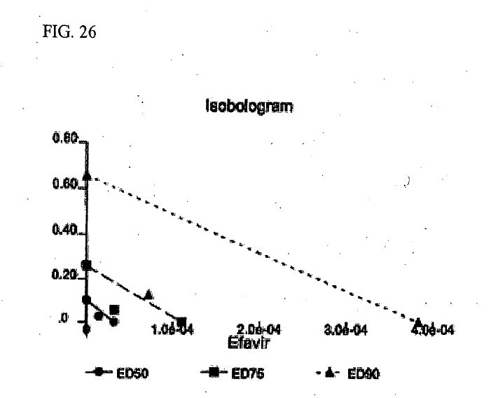

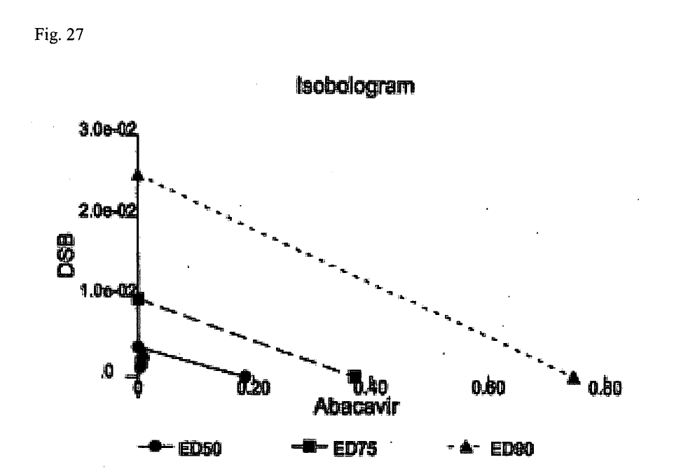

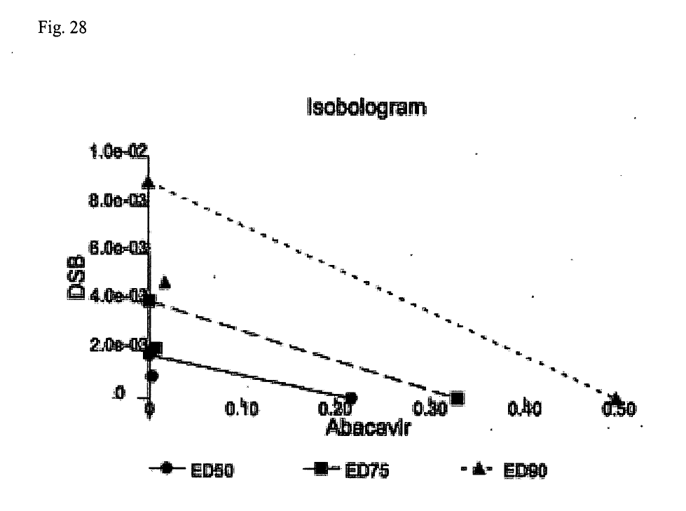

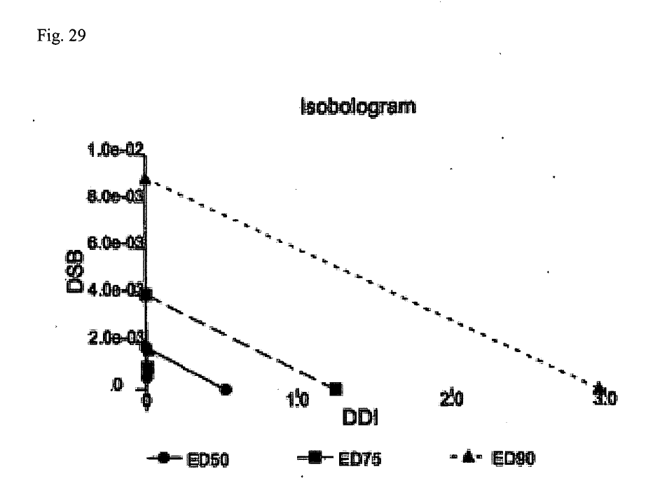

CalcuSyn was used to generate isobolograms depicted in FIGS. 15-29. (CalcuSyn published by Biosoft, version 1.1, copyright 1996.)

Determination of Synergism and Antagonism and Construction of Isobolograms.

The CI was calculated by the Chou-Talalay equation, which takes into account both potency (Dm or IC50) and the shape of the dose effect curve “m”. The general equation for the classic isobologram for mutually exclusive drugs is given by Equation A:

$$CI = \frac{{( D )}_{1}}{{( Dx )}_{1}} + \frac{{( D )}_{2}}{{( Dx )}_{2}}$$

where (Dx)1 and (Dx)2 are the doses (or concentrations) for D1 (DSBx2NmG) and D2 (a second antiretroviral agent) alone that gives x % inhibition, whereas (D)1 and (D)2 are the doses of DSBx2NmG and the second antiretroviral agent in combination that also inhibited x % (i.e., isoeffective). “Mutually exclusive drugs” are drugs that use the same or a similar mechanism of action.

For “mutually nonexclusive drugs,” as in the present case the equation is modified to be:

$$CI = \frac{{( D )}_{1}}{{( {D}_{x} )}_{1}} + \frac{{( D )}_{2}}{{( {D}_{x} )}_{2}} + \frac{{( D )}_{1} {( D )}_{2}}{{( D )}_{1} {( D )}_{2}}$$

“Mutually nonexclusive drugs” are drugs that use different mechanisms of action, for example, drugs that are noncompetitive inhibitors of each other are “mutually nonexclusive.” Owing to the complexity of whole-cell biological systems, the CalcuSyn program automatically analyzes a data set using both the mutually exclusive and mutually nonexclusive assumptions.

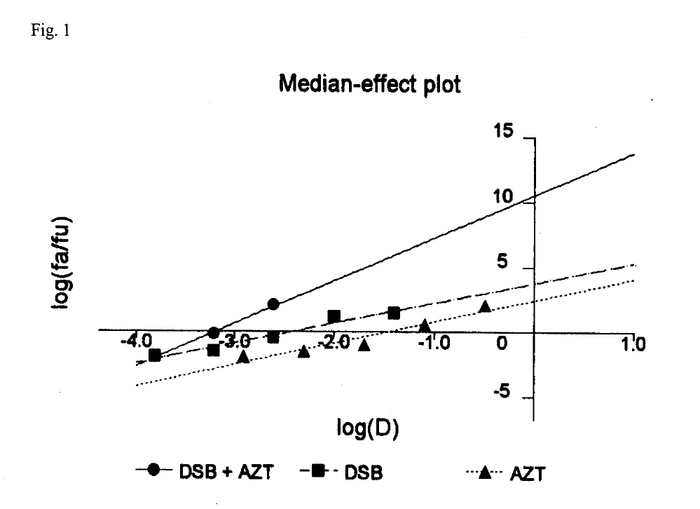

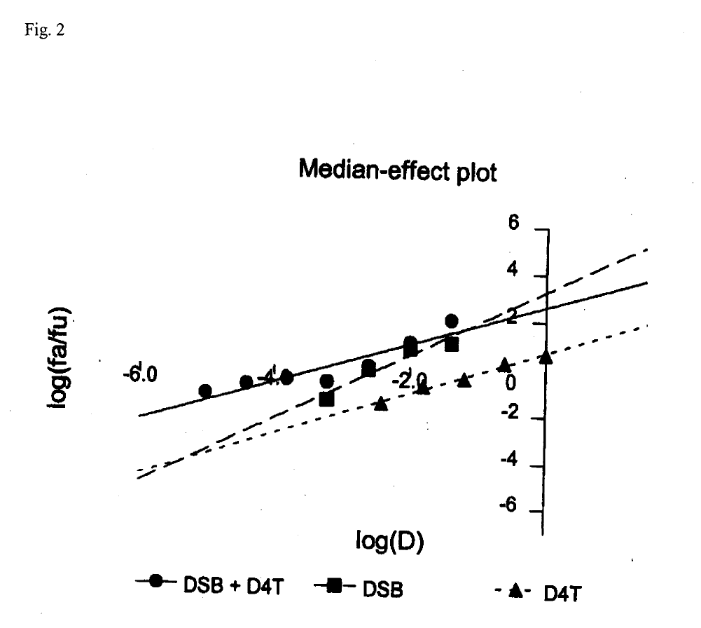

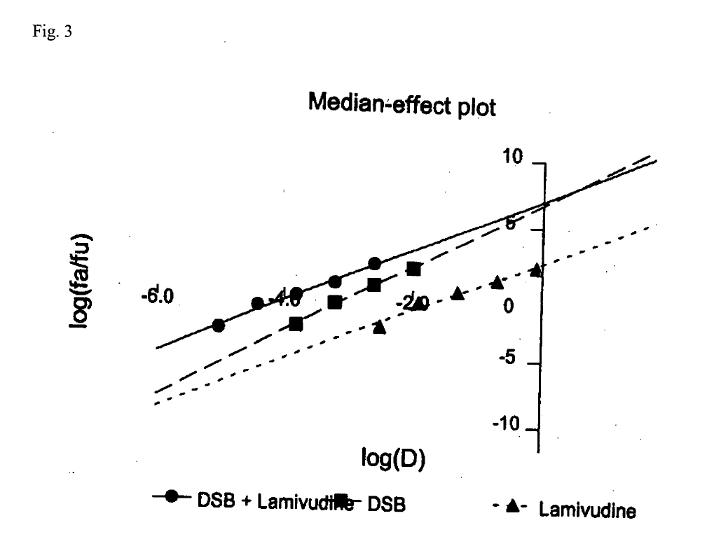

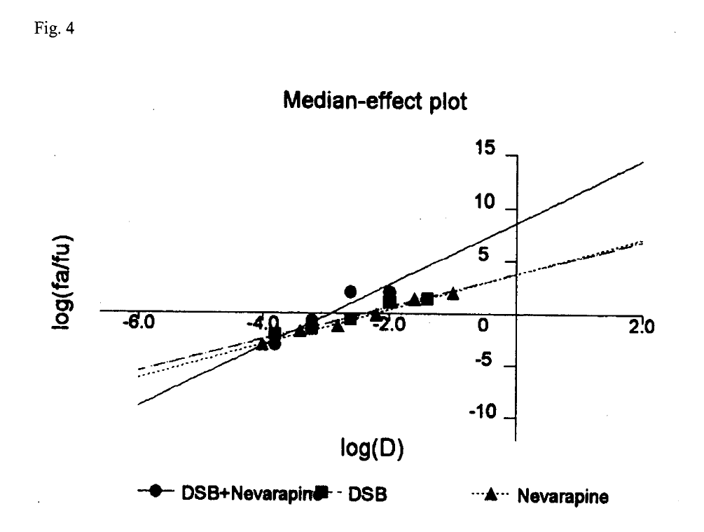

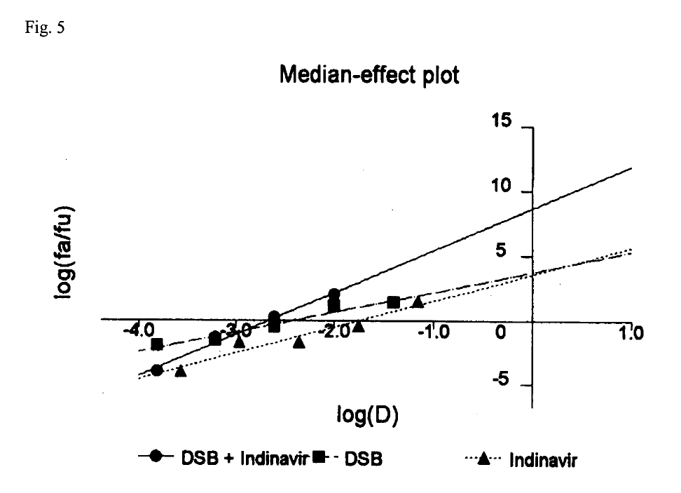

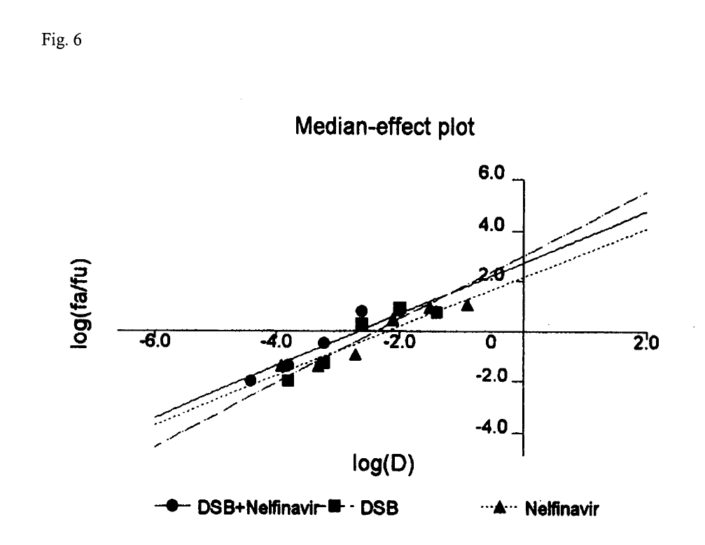

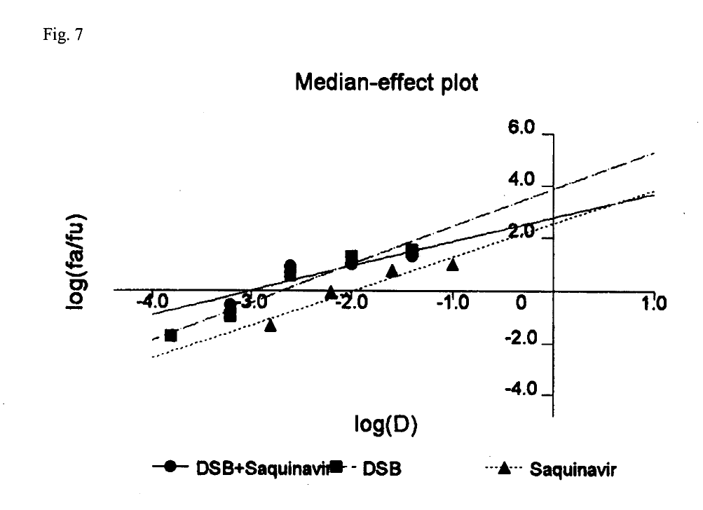

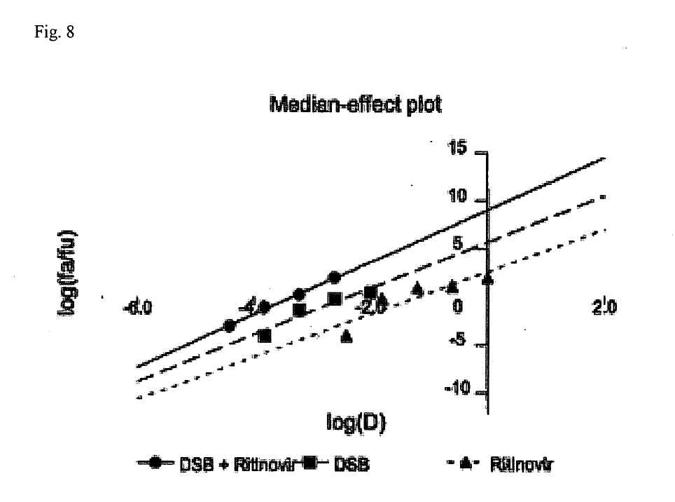

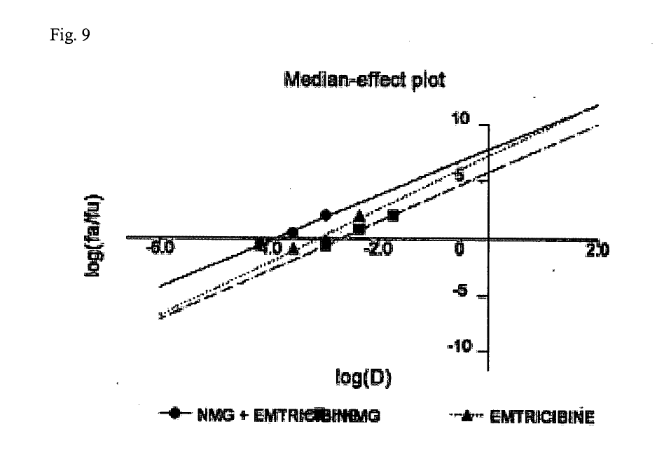

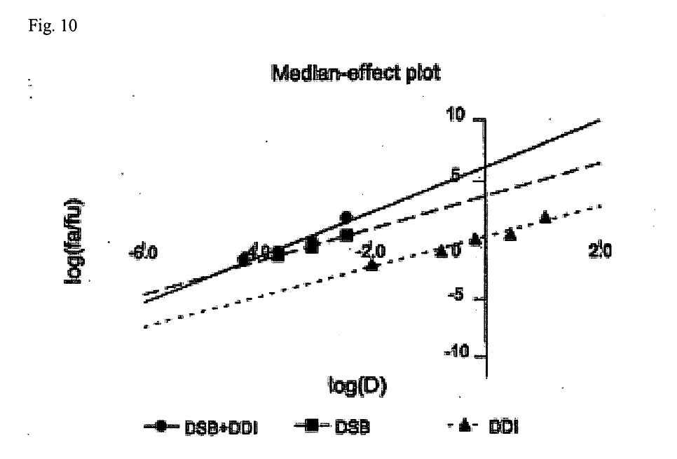

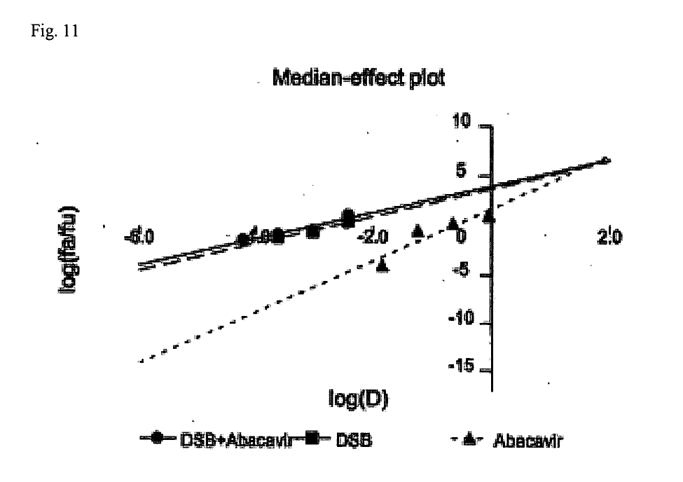

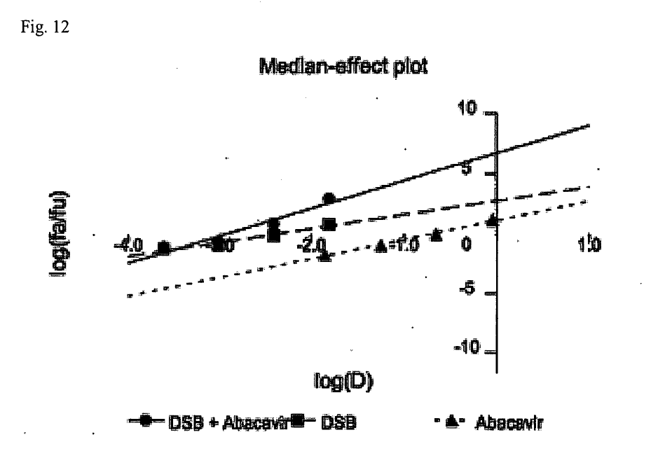

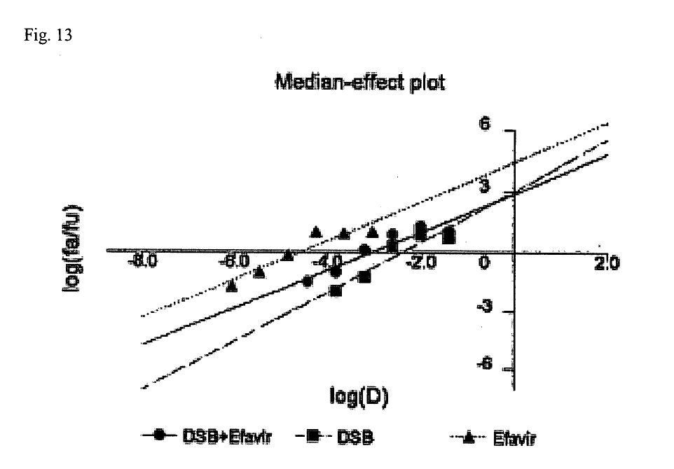

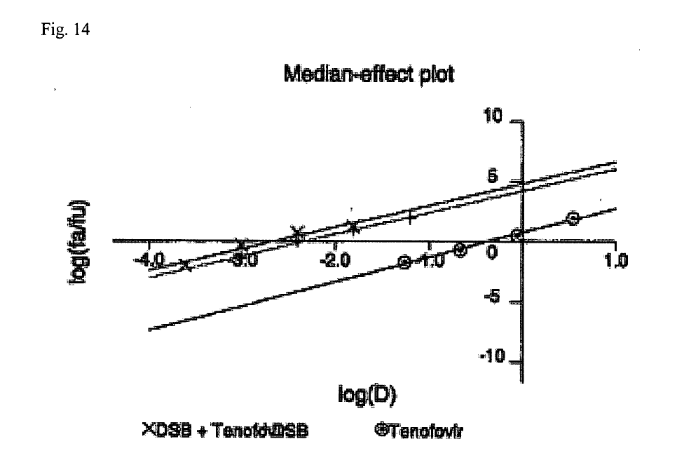

The (Dx)1 or (Dx)2 can be readily calculated from the median-effect equation:

$$Dx = {Dm [ \frac{fa}{( 1 - fa )} ]}^{1 / m}$$

where Dm is the median-effect dose that is obtained from the antilog of the X-intercept of the median-effect plot, X-log (D) versus Y=log [a/(1−a)] or Dm=10−(Y−intercept)/m, and m is the slope of the median-effect plot. Median-effect data are represented by FIGS. 1-14.

CalcuSyn computer software provides for calculation of m, Dm, Dx, and CI values. From (Dm)1, (Dx)2, and D1+D2, the isobolograms can be automatically constructed based on Equation A. Isobologram data are represented by FIGS. 15-29.

The anti-proliferative interactions between DSBx2NmG and 18 compounds representative of 3 common antiretroviral drug classes were analyzed with the isobologram and Chou and Talalay methods, which assess the presence of synergy, additivity, and antagonism. Each of the 18 combinations detailed in Table 2 exhibit surprising co-action or synergy. As a result of such synergism or co-action, a dose-reduction for each agent of a combination can be accomplished over a wide range of drug-effect levels without sacrificing antiretroviral activity.

### EXAMPLE 3

### Formulation of DSB

The DSBx2NmG component can be in liquid form comprising: 41.7 mg/mL of DSBx2NmG salt in 84% water, 4% ethanol, and 12% PEG 400. This illustrative formulation is equivalent to 25 mg/mL of free acid.

Alternatively, the DSBx2NmG component can be in liquid form comprising: 41.7 mg/mL of the DSBx2NmG salt in 10% hydroxy-β-cyclodextrin in water. This illustrative formulation is equivalent to 25 mg/mL of free acid.

### EXAMPLE 4

### Formulation of AZT

The AZT component can be in sustained release capsule form comprising:

| Ingredient | mg/capsule | |
|---|---|---|
| Spherical Cores | | |
| Active Ingredient | 300.0 | |
| Microcrystalline Cellulose | 61.875 | |
| Polyvinylpyrrolidone (PVP) | 13.125 | |
| Purified Water | q.s | |
| Total | 375.0 | mg |
| Sustained Release Coating | | |
| Eudragit NE30D | 5.625 | (Solids) |
| Aquacoat ECD-30 | 5.625 | (Solids) |
| Mannitol | 3.750 | |
| Purified Water | q.s | |
| Total Fill Weight | 390.0 | mg/capsule |

AZT and microcrystalline cellulose are mixed. PVP is dissolved in purified water and added to the blend to granulate. Spherical cores are formed by passing the granules through an extruder and a spheronizer. The spherical cores are dried. The mannitol is dissolved in purified water. The Eudragit NE30D and Aquacoat ECD-30 are added and mixed into the solution. The spherical cores are coated with the suspension, purified talc added to the coated pellets if necessary. The coated pellets are filled in hard-gelatin capsules.

### EXAMPLE 5

### Formulation of Stavudine

The stavudine component can be in tablet form comprising:

| Ingredient | mg/tablet |
|---|---|
| Stavudine | 100 |
| Polyvinylpyrrolidone | 50 |
| (PVP) | |
| Lactose | 125 |
| Magnesium stearate | 25 |
| Total | 300.0 mg |

The stavudine is granulated by a solution of PVP in ethanol, blended with the excipients and compressed using punches to suit

### EXAMPLE 6

### Formulation of Lamivudine

The lamivudine component can be in tablet form comprising:

| Ingredient | mg/tablet |
|---|---|
| Lamivudine | 100.0 |
| Potassium Clavulanate | 62.5 |
| Magnesium Stearate | 17.5 |
| Citric Acid Anhydrous | 48.0 |
| Sodium Bicarbonate | 62.5 |
| Silica Gel Dessicant | 37.5 |
| PVP Cross-Linked Dried | 72.0 |
| Microcrystalline cellulose | 150.0 |
| Total | 550.0 mg |

### EXAMPLE 7

### Formulation of Nevirapine

The nevirapine component can be in tablet form comprising:

| Ingredient | mg/tablet |
|---|---|
| Nevirapine | 200.0 |
| Lactose | 146.0 |
| Hydroxypropyl methylcellulose 2208 | 66.0 |
| Hydroxypropyl cellulose | 50.9 |
| Magnesium stearate | 2.8 |
| Colloidal anhydrous silica | 5.7 |
| Talc | 28.6 |
| Total: | 500.0 mg |

### EXAMPLE 8

### Formulation of Indinavir

The indinavir component can be in tablet form comprising:

| Ingredient | mg/tablet |
|---|---|
| Indinavir Sulfate | 375.0 |
| Anhydrous Lactose | 120.1 |
| Magnesium Stearate | 4.9 |
| Total: | 500.0 mg |

Indinavir sulfate, anhydrous lactose and 50% of the magnesium stearate are blended in a suitable size ribbon mixer for approximately 10 minutes at 20 rpm for a total of approximately 200 revolutions. The resulting powder is fed to a roller compactor for operation at 4 to 10 tons force at a speed of 7 to 15 rpm and a feed speed of 10 to 60 rpm. After the roller compaction step, the compacts are milled using a 0.062 inch screen at a speed of 2000 to 2500 rpm. After milling, the remaining magnesium stearate is used to lubricate the granules in a suitable size ribbon mixer for five minutes at 20 rpm for a total of approximately 100 revolutions. The lubricated granules are encapsulated and the finished capsules are dedusted.

### EXAMPLE 9

### Formulation of Nelfinavir

The nelfinavir component can be in tablet form comprising:

| Ingredient | mg/tablet |
|---|---|
| Nelfinavir | 200.0 |
| Lactose anhydrous | 70.0 |
| Hydroxyethylcellulose | 190.0 |
| Cetostearyl alcohol | 35.0 |
| Talc | 3.0 |
| Magnesium Stearate | 2.0 |
| Purified Water | q.s. |
| Total: | 400.0 mg |

### EXAMPLE 10

### Formulation of Saquinavir

The saquinavir component can be in tablet form comprising:

| Ingredient | mg/tablet |
|---|---|
| Saquinavir | 200.0 |
| Sodium lauryl sulfate | 12.0 |
| Microcrystalline | 120.0 |
| cellulose | |
| Sodium starch | 120.0 |
| glycolate | |
| Lactose, hydrous | 42.0 |
| Magnesium stearate | 6.0 |
| Total: | 500.0 mg |

The saquinavir, sodium starch glycolate and microcrystalline cellulose are granulated using an aqueous solution of sodium lauryl sulfate. This wet mass is dried in a fluid bed. The resulting dried granulation is milled to achieve the desired particle size distribution. This blend is compressed into tablets.

### EXAMPLE 11

### Formulation of Ritonavir

The ritonavir component can be in capsule form comprising:

| Ingredient | mg/capsule |
|---|---|
| Ritonavir | 200.0 |
| Sodium lauryl sulfate | 5.0 |
| Lactose, hydrous | 101.0 |
| Magnesium stearate | 4.0 |
| Sodium starch glycolate (intragranular) | 80.0 |
| Sodium starch glycolate (extragranular) | 10.0 |
| Total: | 500.0 mg |

The ritonavir and intragranular sodium starch glycolate are mixed. After mixing an aqueous solution of sodium lauryl sulfate is added and the resulting mixture is wet granulated. This wet mass is dried in a fluid bed. The dried granulation may then be milled to achieve a suitable particle size distribution. Once a suitable particle size is achieved, the mixture is blended with the other ingredients. This blend is filled into two piece hard gelatin capsule shells.

What is claimed is:

1. A combination comprising a first maturation inhibitor and an antiretroviral agent selected from the group consisting of a protease inhibitor, a reverse transcriptase inhibitor, an integrase inhibitor, a CCR5 antagonist, a fusion inhibitor, and a second maturation inhibitor in amounts effective for treatment of HIV when used in a combination therapy.

2. The combination of claim 1 wherein the first maturation inhibitor is 3-O-(3′,3′-dimethylsuccinyl)betulinic acid or a pharmaceutically acceptable salt thereof.

3. The combination of claim 1 wherein the antiretroviral agent is a protease inhibitor.

4. The combination of claim 3 wherein the protease inhibitor is selected from the group consisting of ritonavir, lopinavir, saquinavir, amprenavir, fosamprenavir, nelfinavir (AG1343), tipranavir, indinavir, atazanavir, brecanavir, TMC-126, darunavir, mozenavir (DMP-450), JE-2147 (AG1776), L-756423 (R-944), KNI-272, DPC-681, DPC-684, SC-52151, BMS 186318, SC-55389a, DMP-323, KNI-227, KIN-272, L697639, PL-100, PPL-100, AG-1859, RO-033-4649, GW-0385, DMP-850, DMP-851, Nar-DG-35, and BMS-232632.

5. The combination of claim 4 wherein the protease inhibitor is ritonavir.

6. The combination of claim 4 wherein the protease inhibitor is nelfinavir.

7. The combination of claim 4 wherein the protease inhibitor is saquinavir.

8. The combination of claim 4 wherein the protease inhibitor is tipranavir.

9. The combination of claim 5 wherein the combination further comprises lopinavir.

10. The combination of claim 4 wherein the protease inhibitor is indinavir.

11. The combination of claim 4 wherein the protease inhibitor is fosamprenavir.

12. The combination of claim 4 wherein the protease inhibitor is atazanavir.

13. The combination of claim 4 wherein the protease inhibitor is brecanavir.

14. The combination of claim 1 wherein the antiretroviral agent is a reverse transcriptase inhibitor.

15. The combination of claim 14 wherein the reverse transcriptase inhibitor is selected from the group consisting of nucleoside reverse transcriptase inhibitors, nucleotide reverse transcriptase inhibitors, and non-nucleoside reverse transcriptase inhibitors.

16. The combination of claim 15 wherein the reverse transcriptase inhibitor is a nucleoside reverse transcriptase inhibitor.

17. The combination of claim 16 wherein the nucleoside reverse transcriptase inhibitor is selected from the group consisting of lamivudine, zidovudine, emtricitabine, abacavir, lamivudine, zalcitabine, didanosine, stavudine, dideoxycytidine, azidothymidine, alovudine, amdoxovir, dexelvucitabine, dioxolane thymidine, elvucitabine, AVX754, DPC-817, KP-1461, MIV-210, racemic emtricitabine, GSK640385, and GSK-204937.

18. The combination of claim 16 wherein the nucleoside reverse transcriptase inhibitor is a thiacytidine nucleoside analog reverse transcriptase inhibitor.

19. The combination of claim 18 wherein the thiacytidine nucleoside analog reverse transcriptase inhibitor is emtricitabine.

20. The combination of claim 16 wherein the nucleoside reverse transcriptase inhibitor is AVX754.

21. The combination of claim 15 wherein the reverse transcriptase inhibitor is a nucleotide reverse transcriptase inhibitor.

22. The combination of claim 21 wherein the nucleotide reverse transcriptase inhibitor is tenofovir disoproxil fumarate.

23. The combination of claim 15 wherein the reverse transcriptase inhibitor is a non-nucleoside reverse transcriptase inhibitor.

24. The combination of claim 23 wherein the non-nucleoside reverse transcriptase inhibitor is selected from the group consisting of diaryltriazine reverse transcriptase inhibitors, and diarylpyrimidines reverse transcriptase inhibitors.

25. The combination of claim 24 wherein the non-nucleoside reverse transcriptase inhibitor is a diarylpyrimidine reverse transcriptase inhibitor.

26. The combination of claim 25 wherein the diarylpyrimidine non-nucleoside reverse transcriptase inhibitor is selected from the group consisting of etravirine and R278474.

27. The combination of claim 23 wherein the non-nucleoside reverse transcriptase inhibitor is selected from the group consisting of atevirdine, delavirdine, nevirapine, capravirine, Calanolide A, dioxolane thymidine, BILR 355BS, SJ-3366, MIV-150, GSK-695634, GSK-678248, KP-1212 and TMC-278.

28. The combination of claim 1 wherein the antiretroviral agent is a CCR5 inhibitor.

29. The combination of claim 28 wherein the CCR5 inhibitor is maraviroc

30. The combination of claim 1 wherein the antiretroviral agent is an integrase inhibitor.

31. The combination of claim 30 wherein the integrase inhibitor is raltegravir.

32. The combination of claim 1 wherein the antiretroviral agent is a fusion inhibitor.

33. The combination of claim 32 wherein the fusion inhibitor is enfuvirtide.

34. The combination of claim 1 wherein the first maturation inhibitor is administered substantially simultaneously with the antiretroviral agent selected from the group consisting of a protease inhibitor, a reverse transcriptase inhibitor, an integrase inhibitor, a CCR5 antagonist, a fusion inhibitor, and a second maturation inhibitor.

35. The combination of claim 1 wherein the first maturation inhibitor is administered sequentially with the antiretroviral agent selected from the group consisting of a protease inhibitor, a reverse transcriptase inhibitor, an integrase inhibitor, a CCR5 antagonist, a fusion inhibitor, and a second maturation inhibitor.
